(19) United States
(12) Patent Application Publication
(10) Pub. No.: US 2023/0008538 A1
(43) Pub. Date: Jan. 12, 2023

(54) PROCESS FOR PREPARING NITRILE INTERMEDIATES FOR NITROGEN-CONTAINING CHELATORS

(71) Applicant: Ascend Performance Materials Operations LLC, Houston, TX (US)

(72) Inventors: Huayun Yu, Houston, TX (US); Zhensheng Ding, Houston, TX (US); Ernst Bezemer, Houston, TX (US); Kyle Kissell, Houston, TX (US)

(73) Assignee: Ascend Performance Materials Operations LLC, Houston, TX (US)

(21) Appl. No.: 17/854,591

(22) Filed: Jun. 30, 2022

Related U.S. Application Data
(60) Provisional application No. 63/216,827, filed on Jun. 30, 2021.

(51) Int. Cl. C07C 209/48; C07C 253/22

(52) U.S. Cl. CPC C07C 209/48; C07C 253/22

(57) ABSTRACT

Reaction pathways and conditions for the production of nitrogen-containing chelators, such as a glycine derivative, with reduction of ammonia byproducts. In particular, the present disclosure describes a process for the production of a nitrile intermediate by reacting a tetra-amino compound or dinitrile compound with an aldehyde and a hydrogen cyanide to form the nitrile intermediate. The nitrile intermediate may then be further processed to produce chelators at a high yield and/or a high purity.

### PRIORITY CLAIM

This application claims priority to U.S. Provisional Application No. 63/216,827, filed on Jun. 30, 2021, which is incorporated herein by reference.

### FIELD

The present disclosure relates generally to the production of nitrile intermediates for nitrogen-containing chelators. In particular, the present disclosure relates to reducing by products, and in particular ammonia, and improving yield & conversion to produce the nitrile intermediates.

### BACKGROUND

Chelators, also known as chelating agents, are organic compounds whose structures allow them to form bonds to a metal atom. Because chelators typically form two or more separate coordinate bonds to a single, central metal atom, chelators can be described as polydentate ligands. Chelators often include sulfur, nitrogen, and/or oxygen, which act as electron-donating atoms in bonds with the metal atom.

Chelators are useful in a variety of applications, where their propensity to form chelate complexes with metal atoms is important. Conventional uses of chelators include in nutritional supplements, in medical treatments (e.g., chelation therapy to remove toxic metals from the body), as contrast agents (e.g., in MRI scans), in domestic and/or industrial cleaners and/or detergents, in the manufacture of catalysts, in removal of metals during water treatment, and in fertilizers. For example, chelators play an important role in treatment of cadmium or mercury poisoning, because the chelators can be selected to selectively bind to the metals and facilitate excretion.

Conventional chelators include, for example, aminopolyphosphonates, polycarboxylates, ethylenediaminetetraacetic acid (EDTA), diethylenetriaminepentaacetic acid (DTPA), and nitrilotriacetic acid (NTA). These and other conventional chelators, however, exhibit a number of undesirable properties. Some conventional chelators do not demonstrate adequate activity or stability across a wide pH and/or temperature range. Some conventional chelators exhibit an unacceptably high toxicity. Some conventional chelators do not exhibit adequate solubility in aqueous and/or organic solvents. Some conventional chelators have low biodegradability and present high environmental risk. Thus, the need exists for chelators that exhibit desirable properties, such as activity and/or stability across a wide pH and/or temperature range, low toxicity, adequate solubility, and/or high biodegradability.

Methyl glycine-N,N-diacetic acid and it salts (referred to as MGDA) are known chelating agents having a good biodegradability that are applied in many several applications as alternatives to EDTA and NTA. The known preparations of MGDA have several downsides including cost for purification and starting materials. While Strecker amino acid synthesis offers an acceptable method, in principal, for preparing intermediates, this synthesis is inefficient and not widely used commercially.

U.S. Pat. No. 5,849,950 describes two processes for glycine-N,N-diacetic acid. The first process involves reacting corresponding 2-substituted glycines or 2-substituted glycinonitriles with formaldehyde and hydrogen cyanide in aqueous medium at a pH of from 0 to 11. The second process involves reacting iminodiacetonitrile or iminodiacetic acid with appropriate monoaldehydes and hydrogen cyanide in aqueous medium at a pH of from 0 to 11. Both process described by U.S. Pat. No. 5,849,950 subsequently hydrolyzed the nitrile functionalities which are present, where the starting material used comprises unpurified raw material derived from the industrial synthesis of glycine derivatives or their precursors or of iminodiacetonitrile or iminodiacetic acid, or mother liquors produced in such syntheses. U.S. Pat. No. 6,005,141 describes similar processes for preparing glycine-N,N-diacetic acid derivatives.

U.S. Pat. No. 7,671,234 describes a process for preparing low-by-product, light-color methylglycine-N,N-diacetic acid tri(alkali metal) salt by alkaline hydrolysis of methylglycinediacetonitrile (MGDN), comprising the steps in the sequence (a) to (f): (a) mixing of MGDN with aqueous alkali at a temperature of ≤30° C.; (b) allowing the aqueous alkaline MGDN suspension to react at a temperature in the range from 10 to 30° C. over a period of from 0.1 to 10 h to form a solution; (c) allowing the solution from step (b) to react at a temperature in the range from 30 to 40° C. over a period of from 0.1 to 10 h; (d) optionally allowing the solution from step (c) to react at a temperature in the range from 50 to 80° C. over a period of from 0.5 to 2 h; (e) optionally allowing the solution from step (c) or (d) to react at a temperature in the range from 110 to 200° C. over a period of from 5 to 60 min; (f) hydrolysis and removal of ammonia of the solution obtained in step (c), (d) or (e) by stripping at a temperature of from 90 to 105° C.

Thus, the need exists for improved processes for producing nitrile intermediates for nitrogen-containing chelators that demonstrate both efficiency and cost-effectiveness improvements. In particular, the need exists for process conditions that do not require a separate crystallization step and have an impurity reduction. The resultant nitrile intermediates should have suitable (or improved) stability and activity across a wide pH and/or temperature range, low toxicity, and suitable biodegradability.

### SUMMARY

In one aspect, the present disclosure describes a process for preparing a nitrile intermediate, the process comprising reacting a tetra-amino compound or dinitrile compound with hydrogen cyanide; and/or an aldehyde of the formula R—CHO, where R is (C₁-C₁₀)alkyl, (C₁-C₁₀)haloalkyl, (C₂-C₁₀)alkenyl, or (C₁-C₁₀)alkyl carboxylate, to form a reaction mixture containing a nitrile intermediate and ammonia, adding an amount of formaldehyde to at least a portion of the reaction mixture to react with ammonia to form one or more tetra-amino compounds or dinitrile compounds, and withdrawing the nitrile intermediate. The process may further comprise separating ammonia from the reaction mixture and adding formaldehyde to the separated portion to form a mixture comprising one or more tetra-amino compounds or dinitrile compounds. In some embodiments, the mixture may be returned to the reaction step, i.e. either the reaction of the tetra-amino compound and/or the reaction of the dinitrile compound. In some embodiments, the amount of formaldehyde is a molar amount that does not exceed the molar amount of the tetra-amino compound or dinitrile compound. The reaction mixture may contain up to 20 wt. % of ammonia, and more preferably up to 6 wt. % of ammonia, which may be reduced by adding formaldehyde or formaldehyde and hydrogen cyanide. In some embodiments, hydrogen cyanide may be added along with the formaldehyde to the reaction mixture to form one or more dinitrile compounds. The formaldehyde added may be added as an aqueous solution. In some embodiment, the aqueous solution contains from 2 to 50 wt. % formaldehyde and the balance being water or other suitable lower alcohols. In some embodiments, the tetra-amino compound may be reacted with hydrogen cyanide following by a subsequent reaction with hydrogen cyanide and the aldehyde. In some cases, the second reaction step comprises adding a nitrile intermediate seed to the reaction mixture. In some cases, the amount of nitrile intermediate seed added is less than 1% the theoretical yield of the nitrile intermediate. The tetra-amino compound used in the reaction may have the following formula:

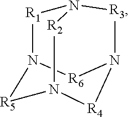

wherein R₁, R₂, R₃, R₄, R₅, and R₆ are independently (C₁-C₅)alkyl or (C₂-C₅)alkenyl. In some embodiments, the tetra-amino compound is hexamethylenetetramine (HMTA) (e.g., 1,3,5,7-tetraazaadamantane). The dinitrile compound used in the reaction may have the following formula:

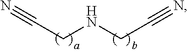

wherein a is from 0 to 5 and b is from 0 to 5. In some embodiments, the dinitrile compound is ((cyanomethyl)amino)acetonitrile, which also may be referred to as iminodiacetonitrile (IDAN). In some embodiments, the nitrile intermediate is alanine-N,N-dinitrile. Preferably, after ammonia byproduct is removed, the process further comprises hydrolyzing the nitrile intermediate forming a glycine-N,N-diacetic acid derivative. In some embodiments, ammonia may be a coproduct with the glycine-N,N-diacetic acid derivative to form a hydrolyzed mixture and further comprising adding an amount of formaldehyde to at least a portion of the hydrolyzed mixture to react with ammonia to form one or more tetra-amino compounds or dinitrile compounds. In some embodiments, the glycine-N,N-diacetic acid derivative has a formula

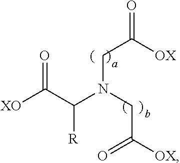

wherein R is (C₁-C₁₀)alkyl, (C₁-C₁₀)haloalkyl, (C₂-C₁₀)alkenyl, or (C₁-C₁₀)alkyl carboxylate, X is hydrogen, an alkali metal, an alkaline earth metal, or ammonium, a is from 0 to 5, and b is from 0 to 5.

In another aspect, the present disclosure describes a process for preparing a nitrile intermediate, the process comprising a first reaction step and second reaction step, followed by adding an amount of formaldehyde to at least a portion of the reaction mixture to react with ammonia to form one or more tetra-amino compounds or dinitrile compounds, and withdrawing the nitrile intermediate. The first reaction step may comprises adding hydrogen cyanide to a tetra-amino compound solution having a pH ranging from 3.0 to 7.0 to form a first intermediate solution, heating and/or chilling the first intermediate solution to the first temperature, maintaining the first intermediate solution at the first temperature, preferably from 35° C. to 75 ° C., for up to 60 minutes, and subsequently further heating the first intermediate solution to a second temperature, preferably from 50° C. to 100° C., greater than the first temperature. In some embodiments, the first intermediate solution is heated to the third temperature, preferably from 35° C. to 75° C., prior to the addition of hydrogen cyanide and aldehyde. In some embodiments, the first reaction step is carried out at a pH from 3.0 to 7.0. The second reaction step may comprise adding hydrogen cyanide and an aldehyde of the formula R—CHO, where R is (C₁-C₁₀)alkyl, (C₁-C₁₀)haloalkyl, (C₂-C₁₀)alkenyl, or (C₁-C₁₀)alkyl carboxylate to the first intermediate solution to form a second intermediate solution; and maintaining the second intermediate solution at a third temperature for from 15 to 250 minutes to form a reaction mixture comprising the nitrile intermediate and ammonia. In some embodiments, the process further comprises adding a nitrile intermediate seed to the second reaction step. In some embodiments, the second reaction step may be carried out at a pH less than 5.0. In one embodiment, the first reaction step and the second reaction step are carried out in one vessel, e.g., the same vessel.

### DETAILED DESCRIPTION

### Introduction

As noted, the present disclosure describes specific reaction pathways and conditions for the production of nitrile intermediates and the nitrogen-containing chelators, e.g., glycine derivatives, produced therefrom. In particular, the present disclosure describes a novel reaction scheme and synergistic combinations of operating parameters for the efficient production of a nitrile intermediate at a high yield and/or purity. The present inventors have developed the reaction scheme, including synergistic combinations of operating parameters, to provide a synthetic route for the production of nitrile intermediates at high yield and/or purity. The nitrile intermediate may then be further processed to produce the nitrogen-containing chelators at a high yield and/or a high purity.

The present disclosure describes a novel process for preparing a nitrile intermediate from a tetra-amino compound and/or dinitrile compound. In one embodiment, in the processes described herein, the nitrile intermediate is formed by a reaction of the tetra-amino compound with a hydrogen cyanide and/or an aldehyde (in an aqueous solution). In another embodiment, in the processes described herein, the nitrile intermediate is formed by a reaction of the dinitrile compound with a hydrogen cyanide and an aldehyde (in an aqueous solution). For purposes forming the nitrile intermediate in either reaction, formaldehyde is not used.

In one embodiment, there is provided a one-step reaction the tetra-amino compound with hydrogen cyanide and the aldehyde as described herein. In another embodiment, there is provided a two-step reaction of the tetra-amino compound that involves an initial reaction with hydrogen cyanide followed by a subsequent reaction with hydrogen cyanide and the aldehyde as described herein. Ammonia may be produced as a byproduct gas in any one of these reactions described further herein. The amount of ammonia produced as a co-product may vary in some cases the ammonia in the reaction mixture may be in amounts of up to 20 wt. %, e.g., up to 15 wt. %, up to 10 wt. %, up to 5 wt %, up to 1 wt %, up to 0.5 wt. % or up to 0.1 wt %. In terms of range, the amount of ammonia may be from 0.001 wt. % to 20 wt. %, e.g., from 0.01 wt. % to 15 wt. %, from 0.01 to 10 wt. %, from 0.01 to 5 wt. %, or from 0.01 wt. % to 1 wt %. Excessively high amounts of ammonia in the reaction mixture may also be reacted by formaldehyde, but problems with the reaction are expected when ammonia is greater than 20 wt. %. When ammonia is separated from the reaction mixture, the ammonia may be concentrated and reacted with the formaldehyde. Reducing the amount of ammonia by a reaction with formaldehyde improves efficiency, conversion, therefore the overall yield of the process.

In some cases, the reacting comprises controlling the addition of the reactants as well as the reaction conditions. For example, in some embodiments, the various reactants may be added and/or combined in a specific order, and the nitrile intermediate seed may be added at specific points in the overall reaction scheme. Controlling the reaction according to the present disclosure may provide for increased yield and/or purity of the nitrile intermediate.

In addition to the improvements in conversion and/or yield, the reaction pathways and conditions described herein may advantageously produce the nitrile intermediate in crystalline form, e.g., without the need for a separate crystallization step. Conventional processes such as Strecker amino acid synthesis, in contrast, are inefficient and produce a nitrile intermediate in non-crystalline form (e.g., as an emulsion), which then requires an inefficient crystallization step. This is typically accomplished by complicated mechanical means, such as complex agitation procedures. The crystallization step reduces the efficiency of the overall reaction and provides further opportunity for the loss of product. Further, the separate crystallization increases production cycle time and requires more equipment. The elimination of the need for crystallizing beneficially increases the efficiency of the reaction. For example, without the need for a separate crystallization step, the nitrile intermediate can be produced and collected more quickly. The crystalline nitrile intermediate also better facilitates conversion to the nitrogen-containing chelator. In addition, removing the crystallization step reduces the costs associated with the production of the nitrogen-containing chelators.

In some cases, a nitrile intermediate seed may be added during the reaction. The nitrile intermediate seed has been found to beneficially promote the formation of the nitrile intermediate in crystalline form. As a result, the (crystalline) nitrile intermediate is surprisingly produced with high purity and/or high yield. Conventional processes do not employ nitrile intermediate seeds, and, as such, require significant additional processing to achieve crystallization (e.g., controlled agitation to produce crystals).

As discussed in detail below, the reacting the tetra-amino compound, hydrogen cyanide, and aldehyde to form the nitrile intermediate may take many forms. The reaction, whether in one-step or two-steps, may comprise combining the reactants in an aqueous solution and allowing the reaction to proceed. In some cases, the reactants are combined substantially simultaneously. In some cases, the reactants are combined in particular order.

### Reactants

### Tetra-Amino Compound

According to the present disclosure, a nitrile intermediate is produced from a tetra-amino compound. The structure of the tetra-amino compound is not particularly limited, and any organic compound having at least four amino functional groups may be used. For example, the tetra-amino compound may comprise a saturated or unsaturated carbon chain having four or more amino functional groups. In some embodiments, the amino functional groups may be moieties of a carbon chain that comprises one or more heteroatoms, such as oxygen, sulfur, or phosphorus. The tetra-amino compound may be aliphatic or aromatic and may be open-chain (e.g., branched-chain, straight-chain) or cyclic (e.g., polycyclic).

In some embodiments, the tetra-amino compound is an aliphatic polycycle having four amino functional groups. For example, the tetra-amino compound may have a chemical structure:

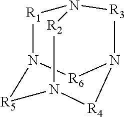

wherein R₁, R₂, R₃, R₄, R₅, and R₆ are independently (C₁-C₅) alkyl or (C₂-C₅)alkenyl. In some embodiments, the tetra-amino compound may have the above chemical structure, R₁, R₂, R₃, R₄, R₅, and R₆ are independently (C₁-C₃)alkyl and more preferably a methylene group. For example, Ri₁R₂, R₃, R₄, R₅, and R₆ may be independently selected from a methylene group, an ethylene group, an n-propylene group, or an isopropylene group. In some embodiments, the tetra-amino compound may have the above chemical structure, wherein at least one of R₁, R₂, R₃, R₄, R₅, and R₆ is a methylene group, e.g., at least two, at least three, or at least four of R₁, R₂, R₃, R₄, R₅, and R₆ are methylene groups. Exemplary tetra-amino compounds according to the above chemical structure include hexamethylenetetramine (HMTA) (e.g., 1,3,5,7-tetraazaadamantane), methyl-tetraazaadamantane, dimethyl-tetraazaadamantane, trimethyl-tetraazaadamantane, tetramethyl-tetraazaadamantane, ethyl-tetraazaadamantane, diethyl-tetraazaadamantane, triethyl-tetraazaadamantane, tetraethyl-tetraazaadamantane, ethyl-methyl-tetraazaadamantane, propyl-tetraazaadamantane, dipropyl-tetraazaadamantane, tripropyl-tetraazaadamantane, tetrapropyl-tetraazaadamantane, and methyl-propyl-tetraazaadamantane. In a preferred embodiment, the tetra-amino compound is HMTA.

In some embodiments, the tetra-amino compound may be dissolved in a solution, e.g., the tetra-amino compound may be a component of a tetra-amino compound solution. For example, the tetra-amino compound may be mixed with and/or dissolved in a solvent. The composition of the tetra-amino compound solution is not particularly limited and may be any solution of the tetra-amino compound. In some embodiments, for example, the tetra-amino compound solution may comprise the tetra-amino compound dissolved in an aqueous solvent, e.g., water, an organic solvent, or a solvent system of both aqueous and organic solvents.

### Aldehyde

The aldehyde may vary widely and many suitable aldehydes are known. In particular, the aldehyde may have a chemical formula R—CHO, where R is (C₁-C₁₀)alkyl, (C₁-C₁₀)haloalkyl, (C₂-C₁₀)alkenyl, or (C₁-C₁₀)alkyl carboxylate. In some embodiments, R of the aldehyde is (C₁-C₁₀)alkyl, e.g., (C₁-C₉)alkyl, (C₁-C₈)alkyl, (C₁-C₇)alkyl, (C₁-C₆)alkyl, or (C₁-C₅)alkyl. In some embodiments, R of the aldehyde is (C₁-C₁₀)haloalkyl, e.g., (C₁-C₉)haloalkyl, (C₁-C₈)haloalkyl, (C₁-C₇)haloalkyl, (C₁-C₆)haloalkyl, or (C₁-C₅)haloalkyl. In some embodiments, R of the aldehyde is unsaturated with one or more double bonds is (C₂-C₁₀)alkenyl, e.g., (C₂-C₉)alkenyl, (C₂-C₈)alkenyl, (C₂-C₇)alkenyl, (C₂-C₆)alkenyl, or (C₂-C₅)alkenyl. In some embodiments, R of the aldehyde is (C₁-C₁₀)alkyl carboxylate, e.g., (C₁-C₉)alkyl carboxylate, (C₁-C₈)alkyl carboxylate, (C₁-C₇)alkyl carboxylate, (C₁-C₆)alkyl carboxylate, or (C₁-C₅)alkyl carboxylate. For example, the aldehyde may comprise a saturated or unsaturated, straight or branched carbon chain, e.g., a terminal carbonyl functional group. Exemplary aldehydes include acetaldehyde, propionaldehyde, butyraldehyde, pentanal, propenal, butenal, pentenal, 2-hexenal, 3-hexenal, crotonaldehyde, formyl ethanoic acid, formyl propionic acid, and formyl butanoic acid. For purposes reacting with the tetra-amino, formaldehyde is not used as an reactant.

As noted above, the order of the addition of the aldehyde to the other reactants may vary widely. In some cases, an aldehyde is added to and/or reacted with the tetra-amino compound (optionally in a heated tetra-amino compound solution) to form a first intermediate solution. For example, the aldehyde may be added to the tetra-amino compound solution, and/or the tetra-amino compound may be added to aldehyde. In some cases, the aldehyde may be added to a solution comprising the tetra-amino compound and the hydrogen cyanide.

The amount of aldehyde used in the reaction is not particularly limited. The amount of aldehyde used may be based on the amount of tetra-amino compound. In some embodiments, for example, an amount of aldehyde is added such that the molar ratio of the aldehyde to the tetra-amino compound is from 0.1:1 to 10:1, e.g., from 0.1:1 to 8:1, from 0.1:1 to 6:1, from 0.1:1 to 4:1, from 0.1:1 to 3:1, from 0.2:1 to 10:1, from 0.2:1 to 8:1, from 0.2:1 to 6:1, from 0.2:1 to 4:1, from 0.2:1 to 3:1, from 0.4:1 to 10:1, from 0.4:1 to 8:1, from 0.4:1 to 6:1, from 0.4:1 to 4:1, from 0.4:1 to 3:1, from 0.5:1 to 10:1, from 0.5:1 to 8:1, from 0.5:1 to 6:1, from 0.5:1 to 4:1, from 0.5:1 to 3:1, from 0.8:1 to 10:1, from 0.8:1 to 8:1, from 0.8:1 to 6:1, from 0.8:1 to 4:1, or from 0.8:1 to 3:1. In terms of lower limits, the molar ratio of the aldehyde to the tetra-amino compound may be greater than 0.1:1, e.g., greater than 0.2:1, greater than 0.4:1, greater than 0.5:1, or greater than 0.8:1. In terms of upper limits, the molar ratio of the aldehyde to the tetra-amino compound may be less than 10:1, e.g., less than 8:1, less than 6:1, less than 4:1, or less than 3:1.

### Hydrogen Cyanide

The hydrogen cyanide (HCN) is added to and/or reacted with one or more of the other reactants. The hydrogen cyanide may be combined with the tetra-amino compound before or after the aldehyde is introduced. In some embodiments, the aldehyde and the hydrogen cyanide are combined with the tetra-amino compound at substantially the same time, e.g., simultaneously or within several minutes of each other. In some embodiments, the hydrogen cyanide is added to and/or reacted with the tetra-amino compound solution.

The HCN, in some instances, may be employed in the form of a solution comprising HCN and the solution may be reacted with the tetra-amino compound (and the aldehyde) as described herein.

The amount of hydrogen cyanide used in the reaction, e.g., the amount hydrogen cyanide added to the tetra-amino compound solution, is not particularly limited. The amount of hydrogen cyanide used may be based on the amount of tetra-amino compound. In some embodiments, for example, an amount of hydrogen cyanide is added such that the molar ratio of the hydrogen cyanide to the tetra-amino compound is from 0.1:1 to 10:1, e.g., from 0.1:1 to 8:1, from 0.1:1 to 6:1, from 0.1:1 to 4:1, from 0.1:1 to 3:1, from 0.2:1 to 10:1, from 0.2:1 to 8:1, from 0.2:1 to 6:1, from 0.2:1 to 4:1, from 0.2:1 to 3:1, from 0.4:1 to 10:1, from 0.4:1 to 8:1, from 0.4:1 to 6:1, from 0.4:1 to 4:1, from 0.4:1 to 3:1, from 0.5:1 to 10:1, from 0.5:1 to 8:1, from 0.5:1 to 6:1, from 0.5:1 to 4:1, from 0.5:1 to 3:1, from 0.8:1 to 10:1, from 0.8:1 to 8:1, from 0.8:1 to 6:1, from 0.8:1 to 4:1, or from 0.8:1 to 3:1. In terms of lower limits, the molar ratio of the hydrogen cyanide to the tetra-amino compound may be greater than 0.1:1, e.g., greater than 0.2:1, greater than 0.4:1, greater than 0.5:1, or greater than 0.8:1. In terms of upper limits, the molar ratio of the hydrogen cyanide to the tetra-amino compound may be less than 10:1, e.g., less than 8:1, less than 6:1, less than 4:1, or less than 3:1.

### Nitrite Intermediate Seed

In some embodiments, the disclosed processes may employ a nitrile intermediate seed during the reaction. The timing of the addition of the nitrile intermediate seed to one or more of the reaction mixtures may vary. The addition of the nitrile intermediate seed has surprisingly been found to greatly improve the preparation of the nitrile intermediate, and subsequently the glycine derivative. In particular, the addition of the nitrile intermediate seed supports the formation of the nitrile intermediate (e.g., by the reaction of the dinitrile compound, the aldehyde, and the hydrogen cyanide) in crystalline form. Said another way, in some cases, the processes described herein produce crystalline nitrile intermediate due to the addition of the nitrile intermediate seed during the reaction. Furthermore, the formation of crystals in situ during the reaction contributes to improved yield and purity of the nitrile intermediate produced by the reaction.

Generally, the nitrile intermediate seed is an organic compound having at least two nitrile, or cyano, functional groups and at least one carboxyl functional group. Exemplary nitrile intermediates include alanine-N,N-diacetonitrile, alanine-N,N-dipropionitrile, alanine-N,N-dibutyronitrile, alanine-N-acetonitrile-N-propionitrile, alanine-N-acetonitrile-N-butyronitrile, ethyl glycine-N,N-diacetonitrile, ethyl glycine-N,N-dipropionitrile, ethyl glycine-N,N-dibutyronitrile, ethyl glycine-N-acetonitrile-N-propionitrile, ethyl glycine-N-acetonitrile-N-butyronitrile, propyl glycine-N,N-diacetonitrile, propyl glycine-N,N-dipropionitrile, propyl glycine-N,N-dibutyronitrile, propyl glycine-N-acetonitrile-N-propionitrile, and propyl glycine-N-acetonitrile-N-butyronitrile

In some embodiments, the chemical composition of the nitrile intermediate seed may be defined in relation to the nitrile intermediate to be formed by the reaction. For example, the nitrile intermediate seed may comprise substantially the same chemical structure (e.g., the same or slightly modified chemical structure) as the nitrile intermediate. Thus, any composition of the nitrile intermediate (discussed in detail below) may be used as the nitrile intermediate seed. The nitrile intermediate seed may be solid of the nitrile intermediate or may be a liquid solution comprising the nitrile intermediate. In some embodiments, for example, the nitrile intermediate seed is solid crystal of the nitrile intermediate.

In the processes described herein, the nitrile intermediate seed is added to a reaction mixture before and/or during the first reaction step or second reaction step. In some embodiments, the nitrile intermediate seed may combined with the dinitrile compound (e.g., the nitrile intermediate seed may be added to the dinitrile compound solution before the addition of both the aldehyde and the hydrogen cyanide). In some embodiments, the nitrile intermediate seed is added to the reaction mixture after the addition of the aldehyde (and before addition of the hydrogen cyanide). For example, the nitrile intermediate seed may be combined with the first intermediate solution (e.g., comprising the dinitrile compound and the aldehyde) to produce a second intermediate solution. In some embodiments, the nitrile intermediate seed is added to the reaction mixture after the addition of the hydrogen cyanide. In some embodiments, the nitrile intermediate seed is added to the reaction mixture at substantially the same time as the aldehyde and/or the hydrogen cyanide.

In some cases, only a small amount of the nitrile intermediate seed is required to produce the effects described herein, but larger amounts are contemplated. The amount of nitrile intermediate seed added to the reaction mixture may be described by reference to the theoretical yield of the nitrile intermediate by the reaction. In some embodiments, the amount of nitrile intermediate seed added to the reaction mixture is less than 1% of the theoretical yield of the nitrile intermediate by the reaction, e.g., less than 0.8%, less than 0.5%, less than 0.2%, less than 0.1%, or less than 0.08%. In terms of lower limits, the amount of nitrile intermediate added to the reaction mixture is greater than 0.0001% the theoretical yield of the nitrile intermediate by the reaction, e.g., greater than 0.0005%, greater than 0.001%, greater than 0.005%, or greater than 0.008%.

The amount of nitrile intermediate seed added to the reaction mixture may also be described by reference to the weight percentage of the nitrile intermediate seed in the reaction mixture (e.g., the weight percentage of the nitrile intermediate seed in the second intermediate solution). In some embodiments, the second intermediate solution comprises from 0.001 wt. % to 1 wt. % of the nitrile intermediate seed, e.g., from 0.001 wt. % to 0.5 wt. %, from 0.001 wt. % to 0.1 wt. %, from 0.001 wt. % to 0.1 wt. %, from 0.001 wt. % to 0.08 wt. %, from 0.005 wt. % to 1 wt. %, from 0.005 wt. % to 0.5 wt. %, from 0.005 wt. % to 0.1 wt. %, from 0.005 wt. % to 0.1 wt. %, from 0.005 wt. % to 0.08 wt. %, from 0.008 wt. % to 1 wt. %, from 0.008 wt. % to 0.5 wt. %, from 0.008 wt. % to 0.1 wt. %, from 0.008 wt. % to 0.1 wt. %, from 0.008 wt. % to 0.08 wt. %, from 0.01 wt. % to 1 wt. %, from 0.01 wt. % to 0.5 wt. %, from 0.01 wt. % to 0.1 wt. %, from 0.01 wt. % to 0.1 wt. %, or from 0.01 wt. % to 0.08 wt. %. In terms of upper limits, the second intermediate solution may comprise less than 1 wt. % nitrile intermediate seed, e.g., less than 0.5 wt. %, less than 0.1 wt. %, or less than 0.08 wt. %.

### Ammonia Reduction

Ammonia may be evolved as a co-product of the nitrile formation. In one embodiment, one mole of ammonia is produced for each mole of tetra-amino compound reacted. This can lead to significant amounts of ammonia in the process. This ammonia may be wasted as gas release or may react with other reagents to form other impurities. In some embodiments, the amount of ammonia in the reaction mixture may be up to 20 wt. %, e.g., up to 15 wt. %, up to 10 wt. %, up to 9 wt. %, up to 8 wt. %, up to 7 wt. %, and more preferably up to 6 wt. %. In terms of ranges, the amount of ammonia may be from 0.5 wt. % to 20 wt. %, e.g., from 0.5 wt. % to 15 wt. %, from 0.5 wt. % to 10 wt. %, from 0.5 wt. % to 9 wt. %, from 0.5 wt. % to 8 wt. %, from 0.5 wt. % to 7 wt. %, and more preferably from 0.5 wt. % to 6 wt. %.

In one embodiment, to reduce ammonia an effective amount of formaldehyde is used. Ammonia can be reduced in the reaction mixture itself or may be separated from the reaction mixture and reduced. One advantageous benefit of using formaldehyde is that the ammonia is reacted under conditions sufficient to yield an tetra-amino reactant, and in particular hexamethylenetetramine. This has the benefit of increasing yield and production rates, while at the same time reducing byproduct formation. In one embodiment, the amount of formaldehyde added is in a molar amount that does not exceed the molar amount of the tetra-amino compound or dinitrile compound. The amount of formaldehyde may be added on a molar basis that is from 0.5 to 2 times the molar amount of the tetra-amino compound or dinitrile compound, e.g., from 0.5 to 1.8, from 0.5 to 1.6, from 0.5 to 1.4, from 0.5 to 1.2, from 0.5 to 1.1, from 0.5 to 1.0, or from 0.5 to 0.9.

The amount of ammonia in the reaction mixture may be reduced by at least 10%, e.g., at least 20%, at least 30%, at least 35%, at least 40%, at least 50%, or at least 75%. In some embodiments, there may be a complete reduction in ammonia. In terms of ranges the amount of ammonia reduced is from 10% to 100%, e.g., from 10% to 95%, from 10 to 90%, from 20% to 90% or from 30% to 90%. Such reduction can further reduce ammonia in the vent gases as well as product.

In one embodiment, formaldehyde was added in a solution comprising from 2 to 60 wt. % formaldehyde, e.g., from 4 to 60 wt. % formaldehyde, from 4 to 55 wt. % formaldehyde, from 5 to 50 wt. % formaldehyde, from 5 to 40 wt. % formaldehyde, from 10 to 40 wt. % formaldehyde, from 15 to 40 wt. % formaldehyde, or from 15 to 37 wt. % formaldehyde. Highly concentrated solutions of formaldehyde of greater than 60 wt. % are difficult to handle and may increase exposure risks. The solution may comprise water or other suitable lower alcohols, such as methanol, ethanol, propanol and/or butanol, as a balance. In one embodiment, the solutions comprises a low level of polyoxymethylene, such as paraformaldehyde and/or polyacetals, in amount of less than 10 wt. %, e.g., less than 5 wt. %, or less than 1 wt. %, or less than 0.5 wt. % or less than 0.1 wt. %, or less than 0.01 wt. %, and in some preferred embodiments may be substantially free of polyoxymethylenes. The use of lower alcohols may beneficial inhibit or reduce polymerization of the formaldehyde. In other embodiments, the aqueous solutions may be alcohol-free. The reaction of ammonia with the aqueous solution of formaldehyde may be conducted at a low temperature from 0° C. to 40° C., e.g. 0° C. to 35° C., or 5° C. to 35° C.

In other embodiments a gaseous stream comprising formaldehyde may be used to react with ammonia. The gaseous stream may comprise more than 15 vol. % formaldehyde, e.g., more than 20 vol. % formaldehyde, more than 25 vol. % formaldehyde, more than 30 vol. % formaldehyde, or more than 35 vol. % formaldehyde. The upper range for formaldehyde in the gaseous stream may be less than or equal to 75 vol. % formaldehyde, e.g., less than or equal to 70 vol. % formaldehyde, less than or equal to 65 vol. % formaldehyde, less than or equal to 60 vol. % formaldehyde, less than or equal to 55 vol. % formaldehyde, less than or equal to 50 vol. % formaldehyde, less than or equal to 45 vol. % formaldehyde, less than or equal to 40 vol. % formaldehyde, or less than or equal to 35 vol. % formaldehyde. The gaseous stream may also comprise inerts, such as helium, nitrogen, or other noble gases.

### Two-Step Reaction

As noted above, the reaction of the processes described herein is carried out in two steps. The utilization of the two-step mechanism provides for the unexpected benefits mentioned herein. In particular, the two-step reaction scheme, including the operating parameters described herein, produces nitrile intermediates at high yield and/or purity. In a first reaction step, the tetra-amino compound is allowed to react with the hydrogen cyanide. This first reaction step produced a reaction intermediate, which may or may not be separated or purified. In a second reaction step, the reaction intermediate is allowed to react with the aldehyde and hydrogen cyanide to produce the nitrile intermediate. Carrying out the reaction in two steps, as described herein, efficiently produces the reaction intermediate (at least in part) before addition of the aldehyde. Further yield improvements are possible with the ammonia reduction described herein. This improves the yield of the ultimate nitrile intermediate.

Each reaction step may comprise controlling the addition of the reactants as well as the reaction conditions. For example, in some embodiments, the various reactants may be added and/or combined in a specific order, and the nitrile intermediate seed may be added at specific points in the overall reaction scheme. The reaction conditions described herein may further improve the production of the nitrile intermediate, e.g., the purity and/or yield of the nitrile intermediate. In particular, the present disclosure provides temperature and pH conditions, which the present inventors have surprisingly found produce the purity and/or the yield of the nitrile intermediate by the reaction described herein.

Controlling the reaction according to the present disclosure may provide for increased yield and/or purity of the nitrile intermediate.

### First Reaction Step

The process of the present disclosure includes a first reaction step of reacting the tetra-amino compound with hydrogen cyanide at a first temperature followed by a second temperature to form a reaction intermediate.

In some embodiments, the first reaction step comprises providing a tetra-amino compound solution comprising the tetra-amino compound. For example, the process may include dissolving the tetra-amino compound in a solvent to prepare the tetra-amino compound solution. The composition of the tetra-amino compound solution is not particularly limited and may be any solution of the tetra-amino compound. In some embodiments, for example, the tetra-amino compound solution may comprise the tetra-amino compound dissolved in an aqueous solvent, e.g., water. In some embodiments, the tetra-amino compound solution may comprise the tetra-amino compound dissolved in an organic solvent. In some embodiments, the tetra-amino compound solution is a solution of the tetra-amino compound dissolved in a solvent system of both aqueous and organic solvents.

The concentration of the tetra-amino compound solution is not particularly limited. In some embodiments, the tetra-amino compound solution comprises from 1 wt. % to 50 wt. % of the tetra-amino compound, e.g., from 1 wt. % to 45 wt. %, from 1 wt. % to 40 wt. %, from 1 wt. % to 35 wt. %, from 1 wt. % to 30 wt. %, from 4 wt. % to 50 wt. %, from 4 wt. % to 45 wt. %, from 4 wt. % to 40 wt. %, from 4 wt. % to 35 wt. %, from 4 wt. % to 30 wt. %, from 8 wt. % to 50 wt. %, from 8 wt. % to 45 wt. %, from 8 wt. % to 40 wt. %, from 8 wt. % to 35 wt. %, from 8 wt. % to 30 wt. %, from 10 wt. % to 50 wt. %, from 10 wt. % to 45 wt. %, from 10 wt. % to 40 wt. %, from 10 wt. % to 35 wt. %, or from 10 wt. % to 30 wt. %. In terms of lower limits, the tetra-amino compound solution may comprise greater than 1 wt. % of the tetra-amino compound, e.g., greater than 4 wt. %, greater than 8 wt. %, or greater than 10 wt. %. In terms of upper limits, the tetra-amino compound solution may comprise less than 40 wt. % of the tetra-amino compound, e.g., less than 45 wt. %, less than 40 wt. %, less than 35 wt. %, or less than 30 wt. %.

Without being limited by theory, the tetra-amino compound solution may be provided for the reaction at any temperature or may be heated to a target temperature. In some embodiments, the tetra-amino compound solution is provided at room temperature. In some embodiments, the tetra-amino compound is from about 10° C. to about 30° C., e.g., from about 10° C. to about 29° C., from about 10° C. to about 28° C., from about 10° C. to about 27° C., from about 10° C. to about 26° C., from about 10° C. to about 25° C., from about 12° C. to about 30° C., from about 12° C. to about 29° C., from about 12° C. to about 28° C., from about 12° C. to about 27° C., from about 12° C. to about 26° C., from about 12° C. to about 25° C., from about 14° C. to about 30° C., from about 14° C. to about 29° C., from about 14° C. to about 28° C., from about 14° C. to about 27° C., from about 14° C. to about 26° C., from about 14° C. to about 25° C., from about 18° C. to about 30° C., from about 18° C. to about 29° C., from about 18° C. to about 28° C., from about 18° C. to about 27° C., from about 18° C. to about 26° C., from about 18° C. to about 25° C., from about 20° C. to about 30° C., from about 20° C. to about 29° C., from about 20° C. to about 28° C., from about 20° C. to about 27° C., from about 20° C. to about 26° C., from about 20° C. to about 25° C., from about 22° C. to about 30° C., from about 22° C. to about 29° C., from about 22° C. to about 28° C., from about 22° C. to about 27° C., from about 22° C. to about 26° C., or from about 22° C. to about 25° C.

In some embodiments, the first reaction step comprises adjusting the pH of the tetra-amino compound solution. The acidity and/or alkalinity of the reactants (and/or the reaction mixture and/or the various intermediate mixtures) can greatly affect the progress of the reaction described herein. In particular, the reaction of the present disclosure may require an acidic environment (e.g., pH less than 7), and so it may be preferable to adjust the pH of the tetra-amino compound solution prior to the addition of and/or mixture with other reactants. In some embodiments, the tetra-amino compound solution is provided at an approximately neutral pH, e.g., a pH ranging from 3.0 to 9, e.g., from 6 to 8, from 6.5 to 7.5, or from 6.75 to 7.25. Thus, in some cases, the reacting comprises adjusting the pH of the dinitrile compound solution. In some embodiments, the pH can be modified by the addition of a mineral acid, e.g., hydrochloric acid, nitric acid, phosphoric acid, sulfuric acid, boric acid, hydrofluoric acid, hydro bromic acid, per chloric acid, or hydroiodic acid.

In some embodiments, the tetra-amino compound solution is adjusted to a pH ranging from 3.0 to 7.0, e.g., from 3.0 to 6.8, from 3.0 to 6.6, from 3.0 to 6.4, from 3.0 to 6.2, from 3.0 to 6.0, from 3.8 to 7.0, from 3.8 to 6.8, from 3.8 to 6.6, from 3.8 to 6.4, from 3.8 to 6.2, from 3.8 to 6.0, from 4.0 to 7.0, from 4.0 to 6.8, from 4.0 to 6.6, from 4.0 to 6.4, from 4.0 to 6.2, from 4.0 to 6.0, from 4.2 to 7.0, from 4.2 to 6.8, from 4.2 to 6.6, from 4.2 to 6.4, from 4.2 to 6.2, from 4.2 to 6.0, from 4.5 to 7.0, from 4.5 to 6.8, from 4.5 to 6.6, from 4.5 to 6.4, from 4.5 to 6.2, or from 4.5 to 6.3.

In some embodiments, the first reaction step comprises adding the hydrogen cyanide to the tetra-amino compound solution to form a first intermediate solution. The method of adding the hydrogen cyanide is not particularly limited. In some cases, for example, the hydrogen cyanide may be added to the tetra-amino compound solution by a syringe, e.g., a sub-surface syringe. In one embodiment, the hydrogen cyanide is added at a rate from 0.01 g/min to 1 g/min, e.g., from 0.02 g/min to 0.8 g/min, from 0.05 g/min to 0.6 g/min, or from 0.08 g/min to 0.4 g/min. In terms of lower limits, the addition rate may be greater than 0.01 g/min, e.g., greater than 0.02 g/min, greater than 0.05 g/min, or greater than 0.08 g/min. In terms of upper limits, the addition rate may be less than 1 g/min, e.g., less than 0.8 g/min, less than 0.6 g/min, or less than 0.4 g/min.

The temperature of the hydrogen cyanide added to the tetra-amino compound solution is not particularly limited. In some cases, the first reaction step comprises adjusting the temperature of the hydrogen cyanide (or the solution containing the hydrogen cyanide). In some embodiments, the hydrogen cyanide is heated or chilled (e.g., before addition to the tetra-amino compound solution) to a temperature from 0° C. to 40° C., e.g., from 1° C. to 35° C., from 2° C. to 30° C., or from 3° C. to 25° C. Similarly, the pH of the hydrogen cyanide is not particularly limited. In some cases, the first reaction step comprises modifying (e.g., controlling and/or adjusting) the pH of the hydrogen cyanide (e.g., before addition to the tetra-amino compound solution) to a pH from 0.5 to 9.

In some embodiments, the first reaction step comprises heating and/or chilling the first intermediate solution to a first temperature. For example, the first intermediate solution may be heated during and/or after the addition of the hydrogen cyanide. In some embodiments, the first temperature is from 35° C. to 75° C., e.g. from 35° C. to 72° C., from 35° C. to 70° C., from 35° C. to 68° C., from 35° C. to 65° C., from 38° C. to 75° C., from 38° C. to 72° C., from 38° C. to 70° C., from 38° C. to 68° C., from 38° C. to 65° C., from 40° C. to 75° C., from 40° C. to 72° C., from 40° C. to 70° C., from 40° C. to 68° C., from 40° C. to 65° C., from 42° C. to 75° C., from 42° C. to 72° C., from 42° C. to 70° C., from 42° C. to 68° C., or from 42° C. to 65° C. In terms of upper limits, the first temperature may be less than 75° C., e.g., less than 72° C., less than 70° C., less than 68° C., or less than 65° C. In terms of lower limits, the first temperature may be greater than 35° C., e.g., greater than 38° C., greater than 40° C., or greater than 42° C. In some embodiments, the first reaction step comprises maintaining the first intermediate solution at the first temperature for up to 60 minutes, e.g., up to 50 minutes, up to 40 minutes, or up to 30 minutes.

In some embodiments, the first reaction step comprises heating and/or chilling the first intermediate solution to a second temperature. For example, the first intermediate solution may be heated during and/or after the addition of the hydrogen cyanide. In some embodiments, the second temperature is from 50° C. to 100° C., e.g. from 50° C. to 95° C., from 50° C. to 90° C., from 50° C. to 85° C., from 50° C. to 80° C., from 55° C. to 100° C., from 55° C. to 95° C., from 55° C. to 90° C., from 55° C. to 85° C., from 55° C. to 80° C., from 60° C. to 100° C., from 60° C. to 95° C., from 60° C. to 90° C., from 60° C. to 85° C., from 60° C. to 80° C., from 65° C. to 100° C., from 65° C. to 95° C., from 65° C. to 90° C., from 65° C. to 85° C., or from 65° C. to 80° C. In terms of upper limits, the second temperature may be less than 100° C., e.g., less than 95° C., less than 90° C., less than 85° C., or less than 80° C. In terms of lower limits, the second temperature may be greater than 50° C., e.g., greater than 55° C., greater than 60° C., or greater than 65° C. In some embodiments, the first reaction step comprises maintaining the first intermediate solution at the second temperature for up to 60 minutes, e.g., up to 50 minutes, up to 40 minutes, or up to 30 minutes.

In some embodiments, the first reaction step comprises some combination of the above-described conditions and parameters. Said another way, the first reaction step may comprise any combination of the above described temperature, pH, and mixing parameters. In some embodiments, for example, the first reaction step may include providing a tetra-amino compound solution comprising the tetra-amino compound, adjusting the pH of the tetra-amino compound solution to a pH ranging from 3.0 to 7.0, adding the hydrogen cyanide to the tetra-amino compound solution to form a first intermediate solution, heating the first intermediate solution a first temperature, maintaining the first intermediate solution at the first temperature for up to 15 minutes, maintaining the first intermediate solution at the first temperature for up to 15 minutes, heating the first intermediate solution to the second temperature, and/or maintaining the first intermediate solution at the second temperature for up to 60 minutes.

### Reaction Intermediate

The first reaction step may produce a reaction intermediate. The reaction intermediate is not particularly limited and will vary with the reactants (e.g., the tetra-amino compound). Generally, the reaction intermediate is a dinitrile compound, e.g., an organic compound having at least two nitrile, or cyano (—C≡N), functional groups. For example, the dinitrile compound may comprise a saturated or unsaturated carbon chain having two or more nitrile functional groups. In some embodiments, the nitrile functional groups may be moieties of a carbon chain that comprises one or more heteroatoms, such as oxygen, nitrogen, sulfur, or phosphorus. In some embodiments, the reaction intermediate is a compound having the chemical structure:

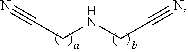

wherein a is from 0 to 5 and b is from 0 to 5. In some embodiments, the reaction intermediate is a dinitrile compound having the above chemical structure, wherein a is 1, and b is 0, 1, 2, 3, 4, or 5. In some embodiments, the dinitrile compound may have the above chemical structure, wherein a is 1 or 2, and b is 0, 1, 2, 3, or 4. In some embodiments, the dinitrile compound may have the above chemical structure, wherein a is 1, 2, or 3, and b is 1, 2, or 3. Exemplary dinitrile compounds according to the above chemical structure include ((cyanomethyl)amino)acetonitrile, ((cyanomethyl)amino)propanenitrile, ((cyanomethyl)amino)butanenitrile, ((cyanomethyl)amino)pentanenitrile, ((cyanoethyl)amino)acetonitrile, ((cyanoethyl)amino)propanenitrile, ((cyanoethyl)amino)butanenitrile, ((cyanoethyl)amino)pentanenitrile, ((cyanopropyl)amino)acetonitrile, ((cyanopropyl)amino)propanenitrile, ((cyanopropyl)amino)butanenitrile, ((cyanopropyl)amino)pentanenitrile, ((cyanobutyl)amino)acetonitrile, ((cyanobutyl)amino)propanenitrile, ((cyanobutyl)amino)butanenitrile, ((cyanobutyl)amino)pentanenitrile, ((cyanopropyl)amino)acetonitrile, ((cyanopropyl)amino)propanenitrile, ((cyanopropyl)amino)butanenitrile, and ((cyanopropyl)amino)pentanenitrile. In a preferred embodiment, the dinitrile compound is ((cyanomethyl)amino)acetonitrile, which also may be referred to as iminodiacetonitrile (IDAN).

### Second Reaction Step

The process of the present disclosure includes a second reaction step of reacting the reaction intermediate with hydrogen cyanide and the aldehyde at a first temperature followed by a second temperature to form the nitrile intermediate. In some cases, the reaction intermediate reacts with aldehyde and/or hydrogen cyanide by Strecker synthesis to produce the nitrile intermediate. Because the components of the first intermediate solution (e.g., the tetra-amino compound, the hydrogen cyanide, the reaction intermediate) may be reactants in the second step, the second reaction step may be carried out in the same vessel as the first reaction step.

In some embodiments, the second reaction step comprises heating and/or chilling the first intermediate solution to a third temperature. For example, the first intermediate solution may be heated during and/or after the completion of the first reaction step. In some embodiments, the third temperature is from 35° C. to 75° C., e.g. from 35° C. to 72° C., from 35° C. to 70° C., from 35° C. to 68° C., from 35° C. to 65° C., from 38° C. to 75° C., from 38° C. to 72° C., from 38° C. to 70° C., from 38° C. to 68° C., from 38° C. to 65° C., from 40° C. to 75° C., from 40° C. to 72° C., from 40° C. to 70° C., from 40° C. to 68° C., from 40° C. to 65° C., from 42° C. to 75° C., from 42° C. to 72° C., from 42° C. to 70° C., from 42° C. to 68° C., or from 42° C. to 65° C. In terms of upper limits, the third temperature may be less than 75° C., e.g., less than 72° C., less than 70° C., less than 68° C., or less than 65° C. In terms of lower limits, the third temperature may be greater than 35° C., e.g., greater than 38° C., greater than 40° C., or greater than 42° C.

In some embodiments, the second reaction step comprises adjusting the pH of the first intermediate solution (e.g., produced in the first reaction step). As noted above, pH can be modified by the addition of a mineral acid, e.g., hydrochloric acid, nitric acid, phosphoric acid, sulfuric acid, boric acid, hydrofluoric acid, hydrobromic acid, perchloric acid, or hydroiodic acid. In some embodiments, the pH of the first intermediate solution is adjusted to a pH ranging from 1.5 to 7.0, e.g., from 1.5 to 6.5, from 1.5 to 6.0, from 1.5 to 5.5, from 2.0 to 7.0, from 2.0 to 6.5, from 2.0 to 6.0, from 2.0 to 5.5, from 2.5 to 7.0, from 2.5 to 6.5, from 2.5 to 6.0, from 2.5 to 5.5, from 3.0 to 7.0, from 3.0 to 6.5, from 3.0 to 6.0, from 3.0 to 5.5.

In some embodiments, the second reaction step comprises adding the aldehyde (or a solution containing the aldehyde) and additional hydrogen cyanide to the first intermediate solution, e.g., to form a second intermediate solution.

The method of adding the aldehyde is not particularly limited. In some cases, for example, the aldehyde may be added to the tetra-amino compound solution by a syringe, e.g., a sub-surface syringe. In one embodiment, the aldehyde is added at a rate from 0.05 mL/min to 10 mL/min, e.g., from 0.1 mL/min to 8 mL/min, from 0.15 mL/min to 5 mL/min, or from 0.2 mL/min to 2 mL/min. In terms of lower limits, the addition rate may be greater than 0.05 mL/min, e.g., greater than 0.1 mL/min, greater than 0.15 mL/min, or greater than 0.2 mL/min. In terms of upper limits, the addition rate may be less than 10 mL/min, e.g., less than 8 mL/min, less than 5 mL/min, less than 2 mL/min, or less than 1 mL/min.

The temperature of the aldehyde added to the first intermediate solution is not particularly limited. In some cases, the second reaction step comprises adjusting the temperature of the aldehyde (or of the solution containing the aldehyde) before addition to the first intermediate solution. In some embodiments, the aldehyde is heated or chilled to a temperature from 1° C. to 40° C., e.g., from 2° C. to 35° C., from 3° C. to 30° C., or from 4° C. to 25° C. Similarly, the pH of the aldehyde is not particularly limited. In some cases, the second reaction comprises modifying (e.g., controlling and/or adjusting) the pH of the aldehyde (e.g., before combination with the tetra-amino compound solution) to a pH from 0.5 to 9.

Likewise, the method of adding the hydrogen cyanide is not particularly limited. In some cases, for example, the hydrogen cyanide may be added to the first intermediate solution by a syringe, e.g., a sub-surface syringe. In one embodiment, the hydrogen cyanide is added at a rate from 0.01 g/min to 1 g/min, e.g., from 0.02 g/min to 0.8 g/min, from 0.05 g/min to 0.6 g/min, or from 0.08 g/min to 0.4 g/min. In terms of lower limits, the addition rate may be greater than 0.01 g/min, e.g., greater than 0.02 g/min, greater than 0.05 g/min, or greater than 0.08 g/min. In terms of upper limits, the addition rate may be less than 1 g/min, e.g., less than 0.8 g/min, less than 0.6 g/min, or less than 0.4 g/min.

The temperature of the hydrogen cyanide added to the first intermediate solution is not particularly limited. In some cases, the second reaction step comprises adjusting the temperature of the hydrogen cyanide (or the solution containing the hydrogen cyanide). In some embodiments, the hydrogen cyanide is heated or chilled to a temperature from 0° C. to 40° C., e.g., from 1° C. to 35° C., from 2° C. to 30° C., or from 3° C. to 25° C. Similarly, the pH of the hydrogen cyanide is not particularly limited. In some cases, the second reaction step comprises modifying (e.g., controlling and/or adjusting) the pH of the hydrogen cyanide (e.g., before addition to the first intermediate solution) to a pH from 0.5 to 9.

In some cases, the second reaction step may comprise adding the nitrile intermediate seed to the reaction mixture (e.g., the first intermediate solution and/or the second intermediate solution). As noted above, a relatively small amount of nitrile intermediate seed is added to the reaction mixture. In some embodiments, the amount of nitrile intermediate seed added to the reaction mixture is less than 1% of the theoretical yield of the nitrile intermediate by the reaction, e.g., less than 0.8%, less than 0.5%, less than 0.2%, less than 0.1%, or less than 0.08%. In terms of lower limits, the amount of nitrile intermediate added to the reaction mixture is greater than 0.0001% the theoretical yield of the nitrile intermediate by the reaction, e.g., greater than 0.0005%, greater than 0.001%, greater than 0.005%, or greater than 0.008%.

In some embodiments, the aldehyde and the hydrogen cyanide are added to the first intermediate solution at the first temperature, as described above. In some embodiments, the first intermediate solution may be heated to the first temperature before, during, and/or after the addition of the aldehyde and/or the hydrogen cyanide.

In some embodiments, the second intermediate solution is maintained at the third temperature to allow the reaction to progress. For example, the second intermediate solution may be maintained at the third temperature for from 15 minutes to 250 minutes, e.g., from 15 minutes to 240 minutes, from 15 minutes to 240 minutes, from 15 minutes to 220 minutes, from 30 minutes to 250 minutes, from 30 minutes to 240 minutes, from 30 minutes to 240 minutes, from 30 minutes to 220 minutes, from 45 minutes to 250 minutes, from 45 minutes to 240 minutes, from 45 minutes to 240 minutes, from 45 minutes to 220 minutes, from 60 minutes to 250 minutes, from 60 minutes to 240 minutes, from 60 minutes to 240 minutes, from 60 minutes to 220 minutes, from 75 minutes to 250 minutes, from 75 minutes to 240 minutes, from 75 minutes to 240 minutes, or from 75 minutes to 220 minutes.

In some embodiments, the second reaction step comprises some combination of the above-described conditions and parameters. Said another way, the second reaction step may comprise any combination of the above described temperature, pH, and mixing parameters. In some embodiments, for example, the second reaction step may include adjusting the pH of the first intermediate solution to a pH ranging from 1.5 to 7.0, adding the hydrogen cyanide and the aldehyde to the first intermediate solution at the second temperature to form a second intermediate solution; and maintaining the second intermediate solution at the second temperature for from 30 to 250 minutes to form the nitrile intermediate.

In some cases, the second reaction step also includes cooling the second intermediate solution. This causes the nitrile intermediate produced by the process to form crystals, which may be harvested (e.g., filtered). In some embodiments, for example, the second intermediate solution is cooled to a temperature less than 25° C., e.g., less than 20° C., less than 15° C., or less than 10° C.

Formaldehyde, as an aqueous solution, is reacted with the ammonia generated by the two-step reaction. In one embodiment, formaldehyde is added to the reaction mixture containing a nitrile intermediate and ammonia after the two-step reaction and the ammonia is reacted out and reduced in concentration. In other embodiments, formaldehyde is contacted with the ammonia that has been separated from the reaction mixture. The ammonia may be reacted out and reduced in concentration, with the subsequent product being recovered and returned to the two-step reaction.

### One-Step Reaction

In another embodiment, the reacting step of the processes described herein may comprise controlling the addition of the reactants as well as the reaction conditions. For example, in some embodiments, the various reactants may be added and/or combined in a specific order, and the nitrile intermediate seed may be added at specific points in the overall reaction scheme. The reaction conditions described herein may further improve the production of the nitrile intermediate, e.g., the purity and/or yield of the nitrile intermediate. In particular, the present disclosure provides temperature and pH conditions, which the present inventors have surprisingly found produce the purity and/or the yield of the nitrile intermediate by the reaction described herein.

Controlling the reaction according to the present disclosure may provide for increased yield and/or purity of the nitrile intermediate.

In some cases, the tetra-amino compound solution may be provided for the reaction at any temperature or may be heated to a target temperature. In some embodiments, the tetra-amino compound solution is provided at room temperature. In some embodiments, the tetra-amino compound is from about 10° C. to about 30° C., e.g., from about 10° C. to about 29° C., from about 10° C. to about 28° C., from about 10° C. to about 27° C., from about 10° C. to about 26° C., from about 10° C. to about 25° C., from about 12° C. to about 30° C., from about 12° C. to about 29° C., from about 12° C. to about 28° C., from about 12° C. to about 27° C., from about 12° C. to about 26° C., from about 12° C. to about 25° C., from about 14° C. to about 30° C., from about 14° C. to about 29° C., from about 14° C. to about 28° C., from about 14° C. to about 27° C., from about 14° C. to about 26° C., from about 14° C. to about 25° C., from about 18° C. to about 30° C., from about 18° C. to about 29° C., from about 18° C. to about 28° C., from about 18° C. to about 27° C., from about 18° C. to about 26° C., from about 18° C. to about 25° C., from about 20° C. to about 30° C., from about 20° C. to about 29° C., from about 20° C. to about 28° C., from about 20° C. to about 27° C., from about 20° C. to about 26° C., from about 20° C. to about 25° C., from about 22° C. to about 30° C., from about 22° C. to about 29° C., from about 22° C. to about 28° C., from about 22° C. to about 27° C., from about 22° C. to about 26° C., or from about 22° C. to about 25° C.

In some cases, the reacting comprises adjusting the temperature of the aldehyde (or of the solution containing the aldehyde). In some embodiments, the aldehyde is heated or chilled (e.g., before combination with the tetra-amino compound solution) to a temperature from 1° C. to 40° C., e.g., from 2° C. to 35° C., from 3° C. to 30° C., or from 4° C. to 25° C. In some cases, the reacting comprises modifying (e.g., controlling and/or adjusting) the pH of the aldehyde (e.g., before combination with the tetra-amino compound solution) to a pH from 0.5 to 9.

As noted, the reacting may comprise adding the aldehyde (or a solution containing the aldehyde) to the tetra-amino compound solution, e.g., to form a first intermediate solution. The method of adding the aldehyde is not particularly limited. In some cases, for example, the aldehyde may be added to the tetra-amino compound solution by a syringe, e.g., a sub-surface syringe. In one embodiment, the aldehyde is added at a rate from 5 g/min to 25 g/min, e.g., from 8 g/min to 22 g/min, from 10 g/min to 20 g/min, or from 12 g/min to 15 g/min. In terms of lower limits, the addition rate may be greater than 5 g/min, e.g., greater than 8 g/min, greater than 10 g/min, or greater than 12 g/min. In terms of upper limits, the addition rate may be less than 25 g/min, e.g., less than 22 g/min, less than 20 g/min, less than 18 g/min, or less than 15 g/min.

In some cases, the reacting comprises adjusting the temperature of the hydrogen cyanide (or the solution containing the hydrogen cyanide). In some embodiments, the hydrogen cyanide is heated or chilled (e.g., before combination with the tetra-amino compound solution) to a temperature from 0° C. to 20° C., e.g., from 0.5° C. to 18° C., from 1° C. to 15° C., or from 1.5° C. to 10° C. In some cases, the reacting comprises modifying (e.g., controlling and/or adjusting) the pH of the hydrogen cyanide (e.g., before combination with the tetra-amino compound solution) to a pH from 0.5 to 9.

As noted, the reacting may comprise adding the hydrogen cyanide (or a solution containing the hydrogen cyanide) to the first intermediate solution, e.g., to form a second intermediate solution. The method of adding the hydrogen cyanide is not particularly limited. In some cases, for example, the hydrogen cyanide may be added to the tetra-amino compound solution by a syringe, e.g., a sub-surface syringe. In one embodiment, the hydrogen cyanide is added at a rate from 0.01 g/min to 1 g/min, e.g., from 0.02 g/min to 0.5 g/min, from 0.05 g/min to 0.3 g/min, or from 0.08 g/min to 0.2 g/min. In terms of lower limits, the addition rate may be greater than 0.01 g/min, e.g., greater than 0.02 g/min, greater than 0.05 g/min, or greater than 0.08 g/min. In terms of upper limits, the addition rate may be less than 1 g/min, e.g., less than 0.5 g/min, less than 0.3 g/min, or less than 0.2 g/min.

In some cases, reacting comprises combining the tetra-amino compound (e.g., the tetra-amino compound solution), the aldehyde, the hydrogen cyanide, and, optionally, the nitrile intermediate seed. In some embodiments, all reactants are combined simultaneously or substantially simultaneously (e.g., within a few minutes of each other). In some embodiments, the reactants are combined in a particular order. In some embodiments, for example, the tetra-amino compound (e.g., the tetra-amino compound solution) and the aldehyde may be combined before the addition of the hydrogen cyanide. In some embodiments, the tetra-amino compound (e.g., the tetra-amino compound solution) and the hydrogen cyanide may be combined before the addition of the aldehyde.

Mixtures, e.g., solutions, formed by the combining of two or more reactants, e.g., the first, second, or third intermediate solutions, may be referred to as reaction mixtures.

The acidity and/or alkalinity of the reactants (and/or the reaction mixture and/or the various intermediate mixtures) can greatly affect the progress of the reaction. In particular, the reaction of the present disclosure may require an acidic environment (e.g., pH less than 7). In some cases, the reacting comprises modifying (e.g., controlling and/or adjusting) the pH of the a reaction mixture (e.g., the first intermediate solution and/or the second intermediate solution). In some embodiments, the pH can be modified by the addition of a mineral acid, e.g., hydrochloric acid, nitric acid, phosphoric acid, sulfuric acid, boric acid, hydrofluoric acid, hydrobromic acid, perchloric acid, or hydriodic acid.

In some embodiments, the pH of one or more reaction mixtures is controlled during the reaction. In some embodiments, the pH is maintained throughout the reaction. For example, the pH of the reaction may be adjusted (e.g., re-adjusted) after the reactants are combined, because the combination of the reactants may affect the pH of the reaction mixture. In some embodiments, the pH of the reaction mixture may be monitored (e.g., with a pH meter), and a mineral acid may be added to the reaction mixture if the measured pH increases with time.

In some embodiments, the reaction mixture is maintained at a pH ranging from 0 to 5, e.g., from 0 to 4.5, from 0 to 4 from 0 to 3.5, from 0 to 3, from 0.5 to 3, from 0.5 to 4.5, from 0.5 to 4 from 0.5 to 3.5, from 0.5 to 3, from 1 to 5, from 1 to 4.5, from 1 to 4 from 1 to 3.5, from 1 to 3, from 1.5 to 5, from 1.5 to 4.5, from 1.5 to 4 from 1.5 to 3.5, or from 1.5 to 3. In terms of lower limits, the pH of the reaction mixture may be maintained at greater than 0, e.g., greater than 0.5, greater than 1, or greater than 1.5 In terms of upper limits, the pH of the reaction mixture may be maintained at less than 5, e.g., less than 4.5, less than 4, less than 3.5, or less than 3.

The temperature of the reaction mixture can also greatly affect the process of the reaction. For example, the temperature of the reaction mixture may affect the solubility of the reactants in solution and/or the rate of the reaction. It is therefore desirable to control the temperature of the reaction mixture. As noted above, for example, each of the reactants (e.g., the tetra-amino compound solution, the acetaldehyde, and/or the hydrogen cyanide) may be heated or chilled before combining. In some embodiments, the temperature of the reaction mixture may also be controlled, or maintained. The method of controlling the temperature of the reaction mixture is not particularly limited. In some embodiments, a mechanical thermal control, e.g., a heat well or a hot plate, may be used to control the temperature of the reaction mixture.

In some embodiments, the temperature of one or more reaction mixtures is controlled during the reaction. In some embodiments, for example, the mixture of the tetra-amino compound (e.g., the tetra-amino compound solution), the aldehyde, the hydrogen cyanide is controlled. In some embodiments, the reaction mixture is heated or chilled during the addition of a reagent. For example, the reaction mixture may be heated during the addition of the aldehyde and/or the hydrogen cyanide.

In some embodiments, the reaction mixture is heated to a temperature from 35° C. and 100° C., e.g., from 35° C. to 95° C., from 35° C. to 90° C., from 35° C. to 85° C., from 35° C. to 80° C., from 40° C. and 100° C., from 40° C. to 95° C., from 40° C. to 90° C., from 40° C. to 85° C., from 40° C. to 80° C., from 45° C. and 100° C., from 45° C. to 95° C., from 45° C. to 90° C., from 45° C. to 85° C., from 45° C. to 80° C., from 50° C. and 100° C., from 50° C. to 95° C., from 50° C. to 90° C., from 50° C. to 85° C., from 50° C. to 80° C., from 55° C. and 100° C., from 55° C. to 95° C., from 55° C. to 90° C., from 55° C. to 85° C., from 55° C. to 80° C., from 60° C. and 100° C., from 60° C. to 95° C., from 60° C. to 90° C., from 60° C. to 85° C., or from 60° C. to 80° C. In terms of lower limits, the reaction mixture may be heated to a temperature greater than 35° C., e.g., greater than 40° C., greater than 45° C., greater than 50° C., greater than 55° C., or greater than 60° C. In terms of upper limits, the reaction mixture may be heated to a temperature less than 100° C., e.g., less than 95° C., less than 90° C., less than 85° C., or less than 80° C. In some cases, the reaction mixture may be heated to a temperature of about 60° C., about 81° C., about 82° C., about 83° C., about 84° C., about 85° C., about 86° C., about 87° C., about 88° C., about 89° C., about 70° C., about 71° C., about 72° C., about 73° C., about 74° C., about 76° C., about 77° C., about 78° C., about 79° C., about 80° C., or a temperature therebetween.

The rate of heating the reaction mixture is not particularly limited. In one embodiment, for example, the reaction mixture is heated at a rate from 1° C./hour to 60° C./hour, e.g., from 1° C./hour to 50° C./hour, from 1° C./hour to 40° C./hour, from 1° C./hour to 30° C./hour, from 1° C./hour to 20° C./hour, from 3° C./hour to 60° C./hour, from 3° C./hour to 50° C./hour, from 3° C./hour to 40° C./hour, from 3° C./hour to 30° C./hour, from 3° C./hour to 20° C./hour, from 6° C./hour to 60° C./hour, from 6° C./hour to 50° C./hour, from 6° C./hour to 40° C./hour, from 6° C./hour to 30° C./hour, from 6° C./hour to 20° C./hour, from 10° C./hour to 60° C./hour, from 10° C./hour to 50° C./hour, from 10° C./hour to 40° C./hour, from 10° C./hour to 30° C./hour, from 10° C./hour to 20° C./hour, from 10° C./hour to 60° C./hour, from 10° C./hour to 50° C./hour, from 10° C./hour to 40° C./hour, from 10° C./hour to 30° C./hour, or from 10° C./hour to 20° C./hour.

The reaction mixture may be maintained at the heated temperature in order to ensure that the reaction runs to completion. In some cases, the reacting includes maintaining the heated temperature for a period of time. In some embodiments, for example, the heated temperature of the reaction mixture may be maintained for from 30 minutes to 180 minutes, e.g., from 30 minutes to 150 minutes, from 30 minutes to 120 minutes, from 30 minutes to 90 minutes, from 45 minutes to 180 minutes, from 45 minutes to 150 minutes, from 45 minutes to 120 minutes, or from 45 minutes to 90 minutes. In terms of lower limits, the heated temperature may be maintained for at least 30 minutes, at least 35 minutes, at least 40 minutes, at least 45 minutes, at least 50 minutes, or at least 55 minutes. In terms of upper limits, the heated temperature may be maintained for less than 180 minutes, e.g., less than 165 minutes, less than 150 minutes, less than 135 minutes, less than 120 minutes or less than 105 minutes. In some cases, the heated temperature is maintained for about 45 minutes, about 50 minutes, about 55 minutes, about 60 minutes, about 65 minutes, about 70 minutes, about 75 minutes, about 80 minutes, about 85 minutes, or about 90 minutes.

In some embodiments, the reacting comprises some combination of the above-described conditions and parameters. Said another way, the reacting may comprise any combination of the above described temperature, pH, and mixing parameters. In some embodiments, for example, the reacting may include providing a tetra-amino compound solution comprising the tetra-amino compound, adjusting the pH of the tetra-amino compound solution to a pH ranging from 0.5 to 4.0, heating the tetra-amino compound solution, adding the aldehyde to the heated tetra-amino compound solution to form a first intermediate solution, adding the nitrile intermediate seed to the first intermediate solution to form a second intermediate solution, adding hydrogen cyanide to the second intermediate solution to form a third intermediate solution, and/or heating the third intermediate solution to form the nitrile intermediate.

In some embodiments, the tetra-amino compound solution is adjusted to a pH ranging from 0.5 to 4, e.g., from 0.5 to 3.8, from 0.5 to 3.6, from 0.5 to 3.4, from 0.5 to 3.2, from 0.5 to 3.0, from 0.8 to 4, from 0.8 to 3.8, from 0.8 to 3.6, from 0.8 to 3.4, from 0.8 to 3.2, from 0.8 to 3.0, from 1.0 to 4, from 1.0 to 3.8, from 1.0 to 3.6, from 1.0 to 3.4, from 1.0 to 3.2, from 1.0 to 3.0, from 1.2 to 4, from 1.2 to 3.8, from 1.2 to 3.6, from 1.2 to 3.4, from 1.2 to 3.2, from 1.2 to 3.0, from 1.5 to 4, from 1.5 to 3.8, from 1.5 to 3.6, from 1.5 to 3.4, from 1.5 to 3.2, or from 1.5 to 3.0.

In some cases, reacting comprises combining the tetra-amino compound (e.g., the tetra-amino compound solution), the aldehyde, the hydrogen cyanide, and/or the nitrile intermediate seed. In some embodiments, all reactants are combined simultaneously or substantially simultaneously (e.g., within a few minutes of each other). In some embodiments, the reactants are combined in a particular order. In some embodiments, for example, the tetra-amino compound (e.g., the tetra-amino compound solution) and the aldehyde may be combined before the addition of the hydrogen cyanide. In some embodiments, the tetra-amino compound (e.g., the tetra-amino compound solution) and the hydrogen cyanide may be combined before the addition of the aldehyde.

Mixtures, e.g., solutions, formed by the combining of two or more reactants, e.g., the first, second, or third intermediate solutions, may be referred to as reaction mixtures

In some embodiments, the pH of one or more reaction mixtures is controlled during the reaction. As noted above, the reaction of the present disclosure may be conducted in an acidic environment. In some embodiments, the pH is maintained throughout the reaction. For example, the pH of the reaction may be adjusted (e.g., re-adjusted) after the reactants are combined, because the combination of the reactants may affect the pH of the reaction mixture. In some embodiments, the pH of the reaction mixture may be monitored (e.g., with a pH meter), and a mineral acid may be added to the reaction mixture if the measured pH increases with time.

In some embodiments, the reaction mixture is maintained at a pH ranging from 0.5 to 4, e.g., from 0.5 to 3.8, from 0.5 to 3.6, from 0.5 to 3.4, from 0.5 to 3.2, from 0.5 to 3.0, from 0.8 to 4, from 0.8 to 3.8, from 0.8 to 3.6, from 0.8 to 3.4, from 0.8 to 3.2, from 0.8 to 3.0, from 1.0 to 4, from 1.0 to 3.8, from 1.0 to 3.6, from 1.0 to 3.4, from 1.0 to 3.2, from 1.0 to 3.0, from 1.2 to 4, from 1.2 to 3.8, from 1.2 to 3.6, from 1.2 to 3.4, from 1.2 to 3.2, from 1.2 to 3.0, from 1.5 to 4, from 1.5 to 3.8, from 1.5 to 3.6, from 1.5 to 3.4, from 1.5 to 3.2, or from 1.5 to 3.0.

The temperature of the reaction mixture can also greatly affect the process of the reaction. For example, the temperature of the reaction mixture may affect the solubility of the reactants in solution and/or the rate of the reaction. It is therefore desirable to control the temperature of the reaction mixture. As noted above, for example, each of the reactants (e.g., the tetra-amino compound solution, the acetaldehyde, and/or the hydrogen cyanide) may be heated before combining. In some embodiments, the temperature of the reaction mixture may also be controlled, or maintained. The method of controlling the temperature of the reaction mixture is not particularly limited. In some embodiments, a mechanical thermal control, e.g., a heat well or a hot plate, may be used to control the temperature of the reaction mixture. Easymax from Mettler Toledo is an example of a suitable, commercially available mechanical thermal control.

In some embodiments, the temperature of one or more reaction mixtures is controlled during the reaction. In some embodiments, for example, the mixture of the tetra-amino compound (e.g., the tetra-amino compound solution), the aldehyde, the hydrogen cyanide, and the nitrile intermediate seed is heated. In some embodiments, the reaction mixture is heated to a temperature (e.g., a first temperature) from 30° C. and 80° C., e.g., from 30° C. to 75° C., from 30° C. to 70° C., from 30° C. to 65° C., from 30° C. to 60° C., from 32° C. and 80° C., from 32° C. to 75° C., from 32° C. to 70° C., from 32° C. to 65° C., from 32° C. to 60° C., from 35° C. and 80° C., from 35° C. to 75° C., from 35° C. to 70° C., from 35° C. to 65° C., from 35° C. to 60° C., from 38° C. and 80° C., from 38° C. to 75° C., from 38° C. to 70° C., from 38° C. to 65° C., from 38° C. to 60° C., from 40° C. and 80° C., from 40° C. to 75° C., from 40° C. to 70° C., from 40° C. to 65° C., from 40° C. to 60° C., from 42° C. and 80° C., from 42° C. to 75° C., from 42° C. to 70° C., from 42° C. to 65° C., or from 42° C. to 60° C. In terms of lower limits, the reaction mixture may be heated to a temperature greater than 30° C., e.g., greater than 32° C., greater than 35° C., greater than 38° C., greater than 40° C., or greater than 42° C. In terms of upper limits, the reaction mixture may be heated to a temperature less than 80° C., e.g., less than 75° C., less than 70° C., less than 65° C., or less than 60° C. In some cases, the reaction mixture may be heated to a temperature of about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 56° C., about 57° C., about 58° C., about 59° C., about 60° C., about 61° C., about 62° C., about 63° C., about 64° C., or about 65° C.

The rate of heating the reaction mixture is not particularly limited. In one embodiment, for example, the reaction mixture is heated at a rate from 1° C./hour to 45° C./hour, e.g., from 1° C./hour to 40° C./hour, from 1° C./hour to 35° C./hour, from 1° C./hour to 30° C./hour, from 1° C./hour to 25° C./hour, from 3° C./hour to 45° C./hour, from 3° C./hour to 40° C./hour, from 3° C./hour to 35° C./hour, from 3° C./hour to 30° C./hour, from 3° C./hour to 25° C./hour, from 6° C./hour to 45° C./hour, from 6° C./hour to 40° C./hour, from 6° C./hour to 35° C./hour, from 6° C./hour to 30° C./hour, from 6° C./hour to 25° C./hour, from 10° C./hour to 45° C./hour, from 10° C./hour to 40° C./hour, from 10° C./hour to 35° C./hour, from 10° C./hour to 30° C./hour, from 10° C./hour to 25° C./hour, from 10° C./hour to 45° C./hour, from 10° C./hour to 40° C./hour, from 10° C./hour to 35° C./hour, from 10° C./hour to 30° C./hour, or from 10° C./hour to 25° C./hour.

The reaction mixture may be maintained at the heated temperature in order to ensure that the reaction runs to completion. In some cases, the reacting includes maintaining the heated temperature for a period of time. In some embodiments, for example, the heated temperature of the reaction mixture may be maintained for from 30 minutes to 180 minutes, e.g., from 30 minutes to 150 minutes, from 30 minutes to 120 minutes, from 30 minutes to 90 minutes, from 45 minutes to 180 minutes, from 45 minutes to 150 minutes, from 45 minutes to 120 minutes, or from 45 minutes to 90 minutes. In terms of lower limits, the heated temperature may be maintained for at least 30 minutes, at least 35 minutes, at least 40 minutes, at least 45 minutes, at least 50 minutes, or at least 55 minutes. In terms of upper limits, the heated temperature may be maintained for less than 180 minutes, e.g., less than 165 minutes, less than 150 minutes, less than 135 minutes, less than 120 minutes or less than 105 minutes. In some cases, the heated temperature is maintained for about 45 minutes, about 50 minutes, about 55 minutes, about 60 minutes, about 65 minutes, about 70 minutes, about 75 minutes, about 80 minutes, about 85 minutes, or about 90 minutes.

In some embodiments, the process described herein includes modulating the temperature of the reaction mixture. For example, the reaction mixture may be heated and/or chilled to the first temperature and maintained at that temperature (as described above), and afterward the reaction mixture may be heated and/or chilled to a second temperature. In some cases, the temperature modulation ensures that the reaction is driven to completion. In some cases, the temperature modulation facilitated the formation of crystals.

In embodiments including temperature modulation, the second temperature is not particularly limited. In some embodiments, the second temperature is from 0° C. to 40° C., e.g., from 0° C. to 30° C., from 0° C. to 25° C., from 0° C. to 20° C., from 1° C. to 40° C., from 1° C. to 30° C., from 1° C. to 25° C., from 1° C. to 20° C., from 2° C. to 40° C., from 2° C. to 30° C., from 2° C. to 25° C., from 2° C. to 20° C., from 3° C. to 40° C., from 3° C. to 30° C., from 3° C. to 25° C., or from 3° C. to 20° C. In terms of lower limits, the second temperature may be greater than 0° C., e.g., greater than 1° C., greater than 2° C., or greater than 3° C. In terms of upper limits, the second temperature may be less than 40° C., e.g., less than 30° C., less than 25° C., or less than 20° C.

In some embodiments, the reacting comprises some combination of the above-described conditions and parameters. Said another way, the reacting may comprise any combination of the above described temperature, pH, and mixing parameters. In some embodiments, for example, the reacting may include providing a tetra-amino compound solution comprising the tetra-amino compound, adding the aldehyde to the tetra-amino compound solution to form a first intermediate solution, adjusting the pH of the first intermediate solution to between 2.0 and 7.0, adding hydrogen cyanide to the first intermediate solution to form a second intermediate solution, heating the second intermediate solution to the first temperature to form the nitrile intermediate, and/or cooling the second intermediate solution to the second temperature to form crystals of the nitrile intermediate.

The yield of nitrile intermediate produced from the one-step process can be improved through the ammonia reduction. Prior to the ammonia reduction, the one-step process has a yield of nitrile intermediate that is greater than 5 wt. %, and this can be further improved once formaldehyde is added. Formaldehyde, as an aqueous solution, is reacted with the ammonia generated by the one-step reaction. In one embodiment, formaldehyde is added to the reaction mixture containing a nitrile intermediate and ammonia after the one-step reaction and the ammonia is reacted out and reduced in concentration. In other embodiments, formaldehyde is contacted with the ammonia that has been separated from the reaction mixture. The ammonia may be reacted out and reduced in concentration, with the subsequent product being recovered and returned to the one-step reaction.

### Product, Nitrite Intermediate

As discussed above, the first reaction step and the second reaction step produce a nitrile intermediate using either two-step reaction or one-step reaction. The nitrile intermediate is not particularly limited and will vary with the tetra-amino compound. Generally, the nitrile intermediate is an organic compound having at least two nitrile, or cyano, functional groups and at least one carboxyl functional group. In some embodiments, the nitrile intermediate is a compound having the chemical structure:

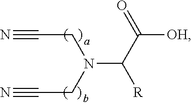

wherein a is from 0 to 5, b is from 0 to 5, and R is (C₁-C₁₀)alkyl, (C₁-C₁₀)haloalkyl, (C₂-C₁₀)alkenyl, or (C₁-C₁₀)alkyl carboxylate. In some embodiments, the nitrile intermediate may have the above chemical structure, wherein a is 1, and b is 0, 1, 2, 3, 4, or 5. In some embodiments, the nitrile intermediate may have the above chemical structure, wherein a is 1 or 2, and b is 0, 1, 2, 3, or 4. In some embodiments, the nitrile intermediate may have the above chemical structure, wherein a is 1, 2, or 3, and b is 1, 2, or 3. In some embodiments, R of the nitrile intermediate is (C₁-C₁₀)alkyl, e.g., (C₁-C₉)alkyl, (C₁-C₈)alkyl, (C₁-C₇)alkyl, (C₁-C₆)alkyl, or (C₁-C₅)alkyl. In some embodiments, R of the nitrile intermediate is (C₁-C₁₀)haloalkyl, e.g., (C₁-C₉)haloalkyl, (C₁-C₈)haloalkyl, (C₁-C₇)haloalkyl, (C₁-C₆)haloalkyl, or (C₁-C₅)haloalkyl. In some embodiments, R of the nitrile intermediate is (C₂-C₁₀)alkenyl, e.g., (C₂-C₁₀)alkenyl, (C₂-C₉)alkenyl, (C₂-C₈)alkenyl, (C₂-C₇)alkenyl, (C₂-C₆)alkenyl, or (C₂-C₅)alkenyl. In some embodiments, R of the nitrile intermediate is (C₁-C₁₀)alkyl carboxylate, e.g., (C₁-C₉)alkyl carboxylate, (C₁-C₈)alkyl carboxylate, (C₁-C₇)alkyl carboxylate, (C₁-C₆)alkyl carboxylate, or (C₁-C₅)alkyl carboxylate. In particular, a and b may correspond to their respective values in the tetra-amino compound, and R may correspond to its respective value in the aldehyde.

Exemplary nitrile intermediates include alanine-N,N-diacetonitrile, alanine-N,N-dipropionitrile, alanine-N,N-dibutyronitrile, alanine-N-acetonitrile-N-propionitrile, alanine-N-acetonitrile-N-butyronitrile, ethyl glycine-N,N-diacetonitrile, ethyl glycine-N,N-dipropionitrile, ethyl glycine-N,N-dibutyronitrile, ethyl glycine-N-acetonitrile-N-propionitrile, ethyl glycine-N-acetonitrile-N-butyronitrile, propyl glycine-N,N-diacetonitrile, propyl glycine-N,N-dipropionitrile, propyl glycine-N,N-dibutyronitrile, propyl glycine-N-acetonitrile-N-propionitrile, and propyl glycine-N-acetonitrile-N-butyronitrile.

As has been discussed, the processes described herein produce the nitrile intermediate in crystalline form. Said another way, crystals of the nitrile intermediate are produced by the described processes, in particular without need for a separate crystallization step. Furthermore, the nitrile intermediate does not form an emulsion and therefore does not require additional mechanical processing (e.g., agitation) to separate. The formation of the nitrile intermediate in crystalline form increases the efficiency of the production process by removing the need for an additional step (and eliminating the time and cost associated therewith).

By carrying out either reaction schemes, i.e., the one-step and/or two-step processes described herein, the nitrile intermediate production is produced in favorable yields. To further improve yield the processes described herein reduces ammonia co-products and obtains the nitrile intermediate at even higher yields. In some embodiments, the nitrile intermediate is formed at a yield greater than 70%, e.g., greater than 75%, greater than 80%, greater than 85%, greater than 90%. In terms of upper limits, the nitrile intermediate may be formed at a yield less than 100%, e.g., less than 99.9%, less than 99.5%, less than 99%, or less than 98%.

In terms of the composition of the reaction mixture (e.g., the solution formed after the second reaction step in the two-step process but before chilling to produce crystals), the content of the nitrile intermediate is also relatively high. In some embodiments, the reaction mixture comprises the nitrile intermediate in an amount greater than 80 wt. %, e.g., greater than 85 wt. %, greater than 90 wt. %, or greater than 95 wt. %. The amount of ammonia produced as byproduct may vary in some cases the ammonia in the reaction mixture may be in amounts of up to 20 wt. %, e.g., up to 15 wt. %, up to 10 wt. %, up to 5 wt %, up to 1 wt %, up to 0.5 wt. % or up to 0.1 wt %. The reaction mixture may further comprise small amounts of unreacted reaction intermediate, e.g., as an impurity and/or side product. In some embodiments, for example, the reaction mixture comprises reaction intermediate in an amount less than 5 wt. %, e.g., less than 3 wt. %, less than 2 wt. %, less than 1 wt. %., or less than 0.5 wt. %.

### Further Reaction

Using either two-step reaction or one-step reaction, the present disclosure also provides reaction pathways that include preparing the glycine derivative, e.g., alanine-N,N-diacetic acid, from the nitrile intermediate formed by the processes described herein, e.g., alanine-N,N-dinitrile (methylglycinediacetonitrile). The structure of the glycine derivative is not particularly limited. As its name suggests, the glycine derivative may be a structural derivative of the amino acid glycine. In particular, the glycine derivative may be any organic compound having at least one carboxyl functional group and at least one amino functional group, wherein the carboxyl functional group and the amino functional group are separated by one carbon atom. In some embodiments, the carbon atom separating carboxyl and amino functional groups may be modified with additional moieties. In some embodiments, the nitrogen of the amino functional group may be modified with additional moieties.

In some embodiments, the glycine derivative is an organic compound having two carboxyl-containing functional groups as moieties on the nitrogen atom of the amino functional group. For example, the glycine derivative may have a chemical structure:

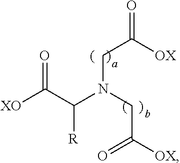

wherein a is from 0 to 5, b is from 0 to 5, and R is (C₁-C₁₀)alkyl, (C₁-C₁₀)haloalkyl, (C₂-C₁₀)alkenyl, or (C₁-C₁₀)alkyl carboxylate. In particular, a and b may correspond to their respective values in the dinitrile compound, and R may correspond to its respective value in the aldehyde. In the above chemical structure, X is hydrogen, an alkali metal, an alkaline earth metal, or ammonium. Exemplary nitrile intermediates include alanine-N,N-diacetic acid, alanine-N,N-dipropionic acid, alanine-N,N-dibutyric acid, alanine-N-acetic acid-N-propionic acid, alanine-N-acetic acid-N-butyric acid, ethyl glycine-N,N-diacetic acid, ethyl glycine-N,N-dipropionic acid, ethyl glycine-N,N-dibutyric acid, ethyl glycine-N-acetic acid-N-propionic acid, ethyl glycine-N-acetic acid-N-butyric acid, propyl glycine-N,N-diacetic acid, propyl glycine-N,N-dipropionic acid, propyl glycine-N,N-dibutyric acid, propyl glycine-N-acetic acid-N-propionic acid, and propyl glycine-N-acetic acid-N-butyric acid.

In the processes described herein, the glycine derivative may be formed by converting the nitrile functional groups of the nitrile intermediate to carboxyl functional groups. In particular, the glycine derivative may be formed by hydrolyzing the nitrile intermediate.

Hydrolysis of the nitrile intermediate is not particularly limited and any known method may be used. In accordance with the embodiments ammonia byproduct is preferably removed before the hydrolysis reaction. In some embodiments, the ammonia byproduct may be removed during or after the hydrolysis reaction. Any ammonia generated in the hydrolysis reaction may be subsequently removed using formaldehyde as described herein. In some embodiments, the hydrolysis is carried out in an aqueous solution using a strong acid. In some embodiments, the hydrolysis is carried out in an aqueous solution using a strong base. Suitable strong bases include inorganic bases, such as ammonium hydroxide, calcium hydroxide, lithium hydroxide, magnesium hydroxide, potassium hydroxide, sodium hydroxide, and combinations thereof.

In one embodiment, ammonia is a coproduct with the glycine-N,N-diacetic acid derivative to form a hydrolyzed mixture and further comprising adding an amount of formaldehyde, such as up to 20 wt. % of ammonia basis on the total weight of the hydrolyzed mixture, e.g., up to 10 wt %., up to 5 wt. %, up to 2 wt. % or up to 1 wt. %, to at least a portion of the hydrolyzed mixture to react with ammonia to form one or more tetra-amino compounds or dinitrile compounds. The tetra-amino compounds or dinitrile compounds may be recovered and reused in the formation of the nitrile intermediate.

The hydrolysis produces the glycine derivative at high yield. In some embodiments, the glycine derivative is formed at a yield greater than 60%, e.g., greater than 65%, greater than 70%, greater than 85%, greater than 90%. In terms of upper limits, the glycine derivative may be formed at a yield less than 100%, e.g., less than 99%, less than 98%, or less than 95%.

### Methyl Glycine-N,N-diacetic Acid (MGDA) Synthesis

In one embodiment, there is an overall reaction to synthesize MGDA from HTMA using the following nitrile reactions (reactions I & II), followed ammonia utilization using either reactions III, IV or both and a hydrolysis reaction (V) to yield MGDA.

HMTA+6 HCN→3 IDAN+NH₃ (I)

IDAN+CH₃CHO+HCN₄→MGDN+H₂O (II)

NH₃+HCHO₄→HMTA (III)

NH₃+HCHO+HCN→IDAN+H₂O (IV)

MGDN+3 NaOH→MGDA+3 NH₃ (V)

The products of reaction (III) may be returned to reaction (I) and products of reaction (IV) may be returned to reaction (II). The ammonia is also a coproduct in reaction (V), and the ammonia may also be utilized to produce HMTA or IDAN as desired using reactions (III) or (IV). In one embodiment, reactions (I) and (II) may be carried out in one vessel, e.g., the same vessel. In another embodiment, reactions (I), (II), and (III) may be carried out in one vessel. In another embodiment, reactions (I), (II), and (IV) may be carried out in one vessel. In still another embodiment, reactions (I), (II), (III) and (IV) may be carried out in one vessel.

It will be appreciated that variants of the above-disclosed and other features and functions, or alternatives thereof, may be combined into many other different systems or applications. Various presently unforeseen or unanticipated alternatives, modifications, variations or improvements therein may be subsequently made by those skilled in the art which are also intended to be encompassed by the following claims or the equivalents thereof.

### EMBODIMENTS

As used below, any reference to a series of embodiments is to be understood as a reference to each of those embodiments disjunctively (e.g., “Embodiments 1-4” is to be understood as “Embodiments 1, 2, 3, or 4”).

Embodiment 1 is a process for preparing a nitrile intermediate, the process comprising reacting a tetra-amino compound or dinitrile compound with hydrogen cyanide; and/or an aldehyde of the formula R—CHO, where R is (C₁-C₁₀)alkyl, (C₁-C₁₀)haloalkyl, (C₂-C₁₀)alkenyl, or (C₁-C₁₀)alkyl carboxylate, to form a reaction mixture containing a nitrile intermediate and ammonia, adding an amount of formaldehyde to at least a portion of the reaction mixture to react with ammonia to form one or more tetra-amino compounds or dinitrile compounds, and withdrawing the nitrile intermediate.

Embodiment 2 is a process for preparing a nitrile intermediate, the process comprising reacting a tetra-amino compound with hydrogen cyanide; and/or an aldehyde of the formula R—CHO, where R is (C₁-C₁₀)alkyl, (C₁-C₁₀)haloalkyl, (C₂-C₁₀)alkenyl, or (C₁-C₁₀)alkyl carboxylate, to form a reaction mixture containing a nitrile intermediate and ammonia, adding an amount of formaldehyde to at least a portion of the reaction mixture to react with ammonia to form one or more tetra-amino compounds, and withdrawing the nitrile intermediate.

Embodiment 3 is a process for preparing a nitrile intermediate, the process comprising reacting a dinitrile compound with hydrogen cyanide; and/or an aldehyde of the formula R—CHO, where R is (C₁-C₁₀)alkyl, (C₁-C₁₀)haloalkyl, (C₂-C₁₀)alkenyl , or (C₁-C₁₀)alkyl carboxylate, to form a reaction mixture containing a nitrile intermediate and ammonia, adding an amount of formaldehyde to at least a portion of the reaction mixture to react with ammonia to form one or more dinitrile compounds, and withdrawing the nitrile intermediate.

Embodiment 4 is a process of embodiments 1 and 3, wherein hydrogen cyanide is added to the reaction mixture to form one or more dinitrile compounds.

Embodiment 5 is a process of embodiments 1-4, further comprising separating ammonia from the reaction mixture and adding formaldehyde to the separated portion to form a mixture comprising one or more tetra-amino compounds or dinitrile compounds.

Embodiment 6 is a process of embodiments 5, wherein the mixture is returned to the reaction step.

Embodiment 7 is a process of embodiments 1-6, wherein the amount of formaldehyde is a molar amount that does not exceed the molar amount of the tetra-amino compound or dinitrile compound.

Embodiment 8 is a process of embodiments 1-7, wherein the reaction mixture contains up to 20 wt. % of ammonia, and preferably up to 6 wt. % of ammonia.

Embodiment 9 is a process of embodiments 1-8, wherein the formaldehyde is added in an aqueous solution comprising from 2 to 50 wt. % formaldehyde.

Embodiment 10 is a process of embodiments 1-9, wherein the tetra-amino compound is reacted with hydrogen cyanide following by a subsequent reaction with hydrogen cyanide and the aldehyde.

Embodiment 11 is a process of embodiments 1-10, further comprising adding a nitrile intermediate seed and the amount of nitrile intermediate seed added is less than 1% the theoretical yield of the nitrile intermediate.

Embodiment 12 is a process of embodiments 1-11, wherein the tetra-amino compound has a formula:

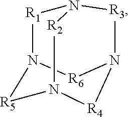

wherein R₁, R₂, R₃, R₄, R₅, and R₆ are independently (C₁-C₅)alkyl or (C₁-C₅)alkenyl.

Embodiment 13 is a process of embodiments 1-12, wherein the dinitrile compound has a chemical structure:

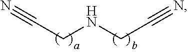

wherein a is from 0 to 5 and b is from 0 to 5.

Embodiment 14 is a process of embodiments 1-13, the nitrile intermediate is alanine-N,N-dinitrile.

Embodiment 15 is a process of embodiments 1-14, further comprising hydrolyzing the nitrile intermediate forming a glycine-N,N-diacetic acid derivative.

Embodiment 16 is a process according to embodiment 15, wherein ammonia is a coproduct with the glycine-N,N-diacetic acid derivative to form a hydrolyzed mixture and further comprising adding an amount of formaldehyde to at least a portion of the hydrolyzed mixture to react with ammonia to form one or more tetra-amino compounds or dinitrile compounds.

Embodiment 17 is a process according to embodiment 15, wherein the glycine-N,N-diacetic acid derivative has a formula

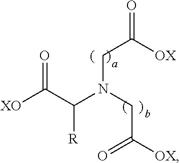

wherein:
R is (C₁-C₁₀)alkyl, (C₁-C₁₀)haloalkyl, (C₂-C₁₀)alkenyl , or (C₁-C₁₀)alkyl carboxylate,
X is hydrogen, an alkali metal, an alkaline earth metal, or ammonium,
a is from 0 to 5, and
b is from 0 to 5.

Embodiment 18 is a process for preparing a nitrile intermediate, the process comprising:
a first reaction step comprising adding hydrogen cyanide to a tetra-amino compound solution having a pH ranging from 3.0 to 7.0 to form a first intermediate solution; heating and/or chilling the first intermediate solution to the first temperature; maintaining the first intermediate solution at the first temperature for up to 60 minutes; and subsequently further heating the first intermediate solution to a second temperature greater than the first temperature; and
a second reaction step comprising adding hydrogen cyanide and an aldehyde of the formula R—CHO, where R is (C₁-C₁₀)alkyl, (C₁-C₁₀)haloalkyl, (C₂-C₁₀)alkenyl, or (C₁-C₁₀)alkyl carboxylate to the first intermediate solution to form a second intermediate solution, and maintaining the second intermediate solution at a third temperature for from 15 to 250 minutes to form a reaction mixture comprising the nitrile intermediate and ammonia;
wherein the process further comprises adding an amount of formaldehyde to at least a portion of the reaction mixture to react with ammonia to form one or more tetra-amino compounds or dinitrile compounds, and withdrawing the nitrile intermediate.

Embodiment 19 is a process according to embodiment 18, wherein the first intermediate solution is heated to the third temperature prior to the addition of hydrogen cyanide and aldehyde.

Embodiment 20 is a process according to embodiments 18 and 19, further comprising adding a nitrile intermediate seed to the second reaction step.

Embodiment 21 is a process according to embodiments 18-20, wherein the first temperature is from 35° C. to 75° C.

Embodiment 22 is a process according to embodiments 18-21, wherein the second temperature is from 50° C. to 100° C.

Embodiment 23 is a process according to embodiments 18-22, wherein the third temperature is from 35° C. to 75° C.

Embodiment 24 is a process according to embodiments 18-23, wherein the first reaction step is carried out at a pH from 3.0 to 7.0.

Embodiment 25 is a process according to embodiments 18-24, wherein the second reaction step is carried out at a pH less than 5.0.

Embodiment 26 is a process according to embodiments 18-25, wherein the first reaction step and the second reaction step are carried out in one vessel.

1. A process for preparing a nitrile intermediate, the process comprising:
reacting a tetra-amino compound or dinitrile compound with hydrogen cyanide; and/or an aldehyde of the formula R—CHO, where R is (C₁-C₁₀)alkyl, (C₁-C₁₀)haloalkyl, (C₂-C₁₀)alkenyl, or (C₁-C₁₀)alkyl carboxylate, to form a reaction mixture containing a nitrile intermediate and ammonia;
adding an amount of formaldehyde to at least a portion of the reaction mixture to react with ammonia to form one or more tetra-amino compounds or dinitrile compounds; and
withdrawing the nitrile intermediate.

2. The process of claim 1, further comprising separating ammonia from the reaction mixture and adding formaldehyde to the separated portion to form a mixture comprising one or more tetra-amino compounds or dinitrile compounds.

3. The process of claim 2, wherein the mixture is returned to the reaction step.

4. The process of claim 1, wherein hydrogen cyanide is added to the reaction mixture to form one or more dinitrile compounds.

5. The process of claim 1, wherein the amount of formaldehyde is a molar amount that does not exceed the molar amount of the tetra-amino compound or dinitrile compound.

6. The process of claim 1, wherein the reaction mixture contains up to 20 wt. % of ammonia.

7. The process of claim 1, wherein the formaldehyde is added in an aqueous solution comprising from 2 to 50 wt. % formaldehyde.

8. The process of claim 1, wherein the tetra-amino compound is reacted with hydrogen cyanide following by a subsequent reaction with hydrogen cyanide and the aldehyde.

9. The process of claim 1, further comprising adding a nitrile intermediate seed.

10. The process of claim 1, wherein the tetra-amino compound has a formula: 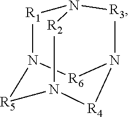
wherein R₁, R₂, R₃, R₄, R₅, and R₆ are independently (C₁-C₅)alkyl or (C₂-C₅)alkenyl.

11. The process according to claim 1, wherein the dinitrile compound has a chemical structure: 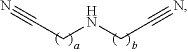
wherein a is from 0 to 5 and b is from 0 to 5.

12. The process according to claim 1, wherein the nitrile intermediate is alanine-N,N-dinitrile.

13. The process according to claim 1, further comprising hydrolyzing the nitrile intermediate forming a glycine-N,N-diacetic acid derivative.

14. The process according to claim 13, wherein ammonia is a coproduct with the glycine-N,N-diacetic acid derivative to form a hydrolyzed mixture and further comprising adding an amount of formaldehyde to at least a portion of the hydrolyzed mixture to react with ammonia to form one or more tetra-amino compounds or dinitrile compounds.

15. The process of claim 13, wherein the glycine-N,N-diacetic acid derivative has a formula 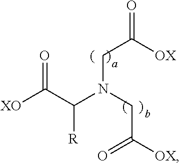
wherein:
R is (C₁-C₁₀)alkyl, (C₁-C₁₀)haloalkyl, (C₂-C₁₀)alkenyl, or (C₁-C₁₀)alkyl carboxylate,
X is hydrogen, an alkali metal, an alkaline earth metal, or ammonium,
a is from 0 to 5, and
b is from 0 to 5.

16. A process for preparing a nitrile intermediate, the process comprising:
a first reaction step comprising:
adding hydrogen cyanide to a tetra-amino compound solution having a pH ranging from 3.0 to 7.0 to form a first intermediate solution;
heating and/or chilling the first intermediate solution to the first temperature;
maintaining the first intermediate solution at the first temperature for up to 60 minutes; and
subsequently further heating the first intermediate solution to a second temperature greater than the first temperature; and
a second reaction step comprising:
adding hydrogen cyanide and an aldehyde of the formula R—CHO, where R is (C₁-C₁₀)alkyl, (C₁-C₁₀)haloalkyl, (C₂-C₁₀)alkenyl, or (C₁-C₁₀)alkyl carboxylate to the first intermediate solution to form a second intermediate solution; and
maintaining the second intermediate solution at a third temperature for from 15 to 250 minutes to form a reaction mixture comprising the nitrile intermediate and ammonia;
wherein the process further comprises
adding an amount of formaldehyde to at least a portion of the reaction mixture to react with ammonia to form one or more tetra-amino compounds or dinitrile compounds; and
withdrawing the nitrile intermediate.

17. The process of claim 16, wherein the first intermediate solution is heated to the third temperature prior to the addition of hydrogen cyanide and aldehyde.

18. The process of claim 16, further comprising adding a nitrile intermediate seed to the second reaction step.

19. The process of claim 16, wherein the first temperature is from 35° C. to 75° C., the second temperature is from 50° C. to 100° C., and third temperature is from 35° C. to 75° C.

20. The process of claim 16, wherein the first reaction step is carried out at a pH from 3.0 to 7.0.

21. The process of claim 16, wherein the second reaction step is carried out at a pH less than 5.0.

22. The process of claim 16, wherein the first reaction step and the second reaction step are carried out in one vessel.
